(19) United States
(12) Patent Application Publication
(10) Pub. No.: US 2008/0057557 A1
(43) Pub. Date: Mar. 6, 2008

(54) METHODS, CASSETTES, GELS AND APPARATUSES FOR ISOLATION AND COLLECTION OF BIOMOLECULES FROM ELECTROPHORESIS GELS

(75) Inventor: Ilana MARGALIT, Ramat Gan (IL)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 11/848,412

(22) Filed: Aug. 31, 2007

Related U.S. Application Data
(60) Provisional application No. 60/829,517, filed on Oct. 13, 2006.
(60) Provisional application No. 60/824,210, filed on Aug. 31, 2006.

(51) Int. Cl. C12N 13/00

(52) U.S. Cl. 435/173.9

(57) ABSTRACT

Electrophoresis systems, assemblies, cassettes and methods for easily, and more effectively and efficiently, isolating a biomolecule band from an electrophoretic gel are provided. The methods use an electrophoresis cassette with at least one loading well and at lest one collection well. A sample containing the biomolecule of interest is placed into at least one loading well and buffer or water is placed in at lest one collection well. An electric field is then applied to drive migration and separation of the sample into different component bands within the gel. When the component of interest is located within at least one collection well, the electric field is terminated and the buffer or water in the collection well is removed, thereby isolating and collecting the sample component of interest.

### CROSS-REFERENCE TO RELATED APPLICATIONS

This application claims benefit of priority to U.S. Provisional Application 60/824,210, entitled “Methods, Cassettes, Gels and Apparatuses for Isolation and Collection of Biomolecules from Electrophoresis Gels”, filed Aug. 31, 2006; and U.S. Provisional Application 60/829,517, entitled “Methods, Cassettes, Gels and Apparatuses for Isolation and Collection of Biomolecules from Electrophoresis Gels”, filed Oct. 13, 2006; each of which is herein incorporated by reference in their entirety.

### FIELD OF THE INVENTION

This invention relates to electrophoresis methods and apparatus for enabling isolation and collection of a band from an electrophoretic gel.

### BACKGROUND OF THE INVENTION

Gel electrophoresis is a common tool for analyzing components of a biological sample to identify new drug candidates or to identify new diagnostic tests, for example. During gel electrophoresis, individual components of a sample, which typically are not individually visible and separated within a biological sample, are separated into individual bands, which can be visualized.

Once a sample, such as a protein sample or a sample of DNA molecules, has been subjected to an electrophoresis procedure, it is often desired to remove a specific sample component band, such as a specific protein or DNA band, from the electrophoresis gel. Methods for collecting sample bands from an electrophoresis gel include cutting out the identified band of interest from the gel, and recovering the sample component of interest from the excised gel, or making a slit into the gel and inserting a cup with an immobilization surface to catch the sample component of interest. However, such methods are inefficient and can lead to undesirable dilution of a sample component. Furthermore, such methods are not well-suited for high-throughput methods using disposable commercial products.

### SUMMARY OF THE INVENTION

Provided herein are electrophoresis systems, assemblies, cassettes and methods for more effective and efficient, isolation of a biomolecule band from an electrophoretic gel. In illustrative embodiments, the electrophoresis systems, assemblies, cassettes and methods include a closed electrophoresis cassette that can be disposable. Therefore, a customer can purchase a closed electrophoresis cassette product from an outside vendor, and use the purchased product to perform methods provided herein.

In one aspect provided herein are methods for isolating a biomolecule or a band from an electrophoresis gel wherein such methods includes the steps of providing or obtaining a closed electrophoresis cassette, loading a sample that includes a biomolecule into at least one loading well through at least one loading aperture of the electrophoresis cassette; applying an electric field between the electrodes of the electrophoresis cassette to drive electrophoretic migration of the biomolecule into a collection well, and removing the liquid containing the biomolecule or band of interest from the collection well through the collection aperture, thereby isolating the biomolecule or band of interest. In such methods the closed electrophoresis cassette includes the following: a separation chamber having walls, wherein at least one of the walls has an array of apertures, including at least one row of loading apertures and at least one row of collection apertures; an electrophoresis gel contained within the separation chamber; an array of well, including at least one row of loading wells and at least one row of collection wells, within the electrophoresis gel, wherein each loading well is accessible through a loading aperture and each collection well is accessible through a collection aperture, and each loading well is aligned with at least one collection well in an electrophoresis lane, and wherein the collection wells are filled with a liquid; and at least two electrodes that include at least one anode and at least one cathode, wherein the rows of wells and apertures (array of wells and apertures) are located between the anodes and cathodes.

In certain embodiments of this aspect the collection wells are filled with water, while in other alternative embodiments the collection wells are filled with buffer.

In other embodiments, the wall having the apertures has an inner surface and an outer surface and the inner surface and the outer surfaces are not in contact with a liquid. In further or alternative embodiments, the closed electrophoresis cassette is not immersed in a running buffer used to drive the electrophoretic separation. In other embodiments, there is no liquid above the electrophoresis gel in contact with a collection well. In other embodiments, there is no fluid communication between the liquid in the collection wells and a running buffer used to drive the electrophoretic separation, while in other embodiments the liquid in the collection wells makes no contact with another liquid in the electrophoresis cassette during the loading or the collecting. In other embodiments, there is no fluid communication between the liquid in the collection wells and another liquid.

In other embodiments, the only liquids within the electrophoresis cassette is the liquid in the collection wells, and optionally liquid in the sample wells. In other embodiments, the liquid in the collection wells is isolated from other liquids that optionally are present within the electrophoresis cassette.

In further or alternative embodiments, the methods of this aspect also include terminating the electric field when the biomolecule is within the collection well. In other embodiments, the methods also include loading the collection well with liquid after the collection step. In other embodiments the methods also include applying the electric field between the electrodes to drive electrophoretic migration of a second biomolecule into the collection well, and in other embodiments the methods also include collecting the second biomolecule from the collection well through the collection aperture. In other embodiments the methods also include terminating the electric field when the second biomolecule is within the collection well. In other embodiments the methods also include reversing the electric field polarity. In other embodiments the methods also include applying the electric field between the electrodes to drive electrophoretic migration of a second biomolecule into a second collection well located in the electrophoresis lane. In other embodiments the methods also include collecting the second biomolecule from the second collection well through a second collection aperture. In other embodiments the methods also include terminating the electric field when the second biomolecule is within the second collection well.

In further or alternative embodiments, the methods of this aspect also include monitoring the location of the biomolecule during application of the electric field. In other embodiments the monitoring does not damage the biomolecules in the sample. In other embodiments the monitoring includes illuminating with UV light. In other embodiments the monitoring includes illuminating with white light, blue light or visible light. In other embodiments the monitoring includes detecting fluorescence. In other embodiments the monitoring is constant. In further or alternative embodiments, the methods also include loading the electrophoresis cassette into a device that combines a means for applying the electric field and a means for monitoring the plurality of bands or biomolecules during electrophoretic transport of the bands or biomolecules.

In further or alternative embodiments, the methods also include monitoring the biomolecule migrating past a marking located on at least one wall of the electrophoresis cassette. In other embodiments the methods also include monitoring the biomolecule migrating past a gradient of markings located on at least one wall of the electrophoresis cassette. In other embodiments such gradients are linear gradients.

In further or alternative embodiments, the electrophoresis cassette also includes a dye. In other embodiments, the sample is associated with a dye prior to loading into the loading wells, while in still other embodiments the sample is associated with a dye after loading into the loading wells. In further or alternative embodiments, the methods also include loading a standard comprising a plurality of known molecular markers. In certain embodiments, such markers include a dye.

In certain embodiments of such methods the biomolecule is DNA or fragments thereof, while in other embodiments the biomolecule is RNA or fragments thereof. In alternative embodiments of such methods the biomolecule is a peptide, while in other embodiments the biomolecule is a protein or fragments thereof. In other embodiments such methods also include loading the biomolecule into a loading well after it is collected. In certain embodiments, such loading wells are located in a different electrophoresis cartridge from the one used to isolate and collect the biomolecule. In other embodiments the methods also include analyzing the biomolecule after collection. In certain embodiments the biomolecule is analyzed by mass spectrometry. In other embodiments the methods also include using the biomolecule in a biochemical process. In certain embodiments the biochemical process is a cloning reaction, while in other embodiments the biochemical process is a ligation reaction. In certain embodiments the biochemical process is restriction enzyme cloning, while in other embodiments the biochemical process is high-throughput recombination cloning. In certain embodiments the biochemical process is TOPO® (Invitrogen Corp., Carlsbad) restriction cloning, while in other embodiments the biochemical process is GATEWAY® (Invitrogen Corp., Carlsbad) recombination cloning. In certain embodiments, the efficiencies of such cloning methods are enhanced 10-1000 fold, while in other embodiments the efficiency is enhanced 10-500 fold. In certain embodiments, the efficiencies of such cloning methods are enhanced 10-100 fold.

In further or alternative embodiments, the electric field used in such methods is a pulsed electric field. In certain embodiments, such pulsed electric fields are generated using pulsed voltage, while in other embodiments such pulsed electric fields are generated using pulsed current. In still other embodiments such pulsed electric fields are generated using pulsed power. In further or alternative embodiments, the electric field used in such methods is a constant electric field. In certain embodiments, such constant electric fields are generated using constant applied voltage, while in other embodiments such constant electric fields are generated using constant applied current. In still other embodiments such constant electric fields are generated using constant applied power.

In further or alternative embodiments, the electrophoresis gel includes agarose, while in other embodiments the electrophoresis gel includes polyacrylamide. In still further embodiments the electrophoresis gel includes agarose and polyacrylamide. In certain embodiments, the width of each loading well in the electrophoresis gel is from 3 mm to 5 mm and the height of each loading well in the electrophoresis gel is from 1 mm to 2.5 mm. In certain embodiments, the width of each collection well in the electrophoresis gel is from 3 mm to 5 mm and the height of each collection well in the electrophoresis gel is from 1 mm to 2.5 mm. In certain embodiments the volume of each loading well in the electrophoresis gel is in the range from 10 μL to 30 μL. In certain embodiments the volume of each collection well in the electrophoresis gel is in the range from 10 μL to 30 μL.

In another aspect provided herein are electrophoresis cassettes for isolating a biomolecule or a band from an electrophoresis gel. Such cassettes include the following: a separation chamber having walls, wherein at least one of the walls has an array of apertures, including at least one row of loading apertures and at least one row of collection apertures; an electrophoresis gel contained within the separation chamber; an array of well, including at least one row of loading wells and at least one row of collection wells, within the electrophoresis gel, wherein each loading well is located underneath a loading aperture and each collection well is located underneath a collection aperture, and each loading well is aligned with at least one collection well in an electrophoresis lane, and wherein the collection wells are filled with a liquid; at least two electrodes, which are at least on anode and at least on cathode, and wherein the rows of wells and apertures are located between the electrodes; and at least one marking on the wall comprising the array apertures, including at least one row of loading apertures and at least one row of collection apertures, wherein the at least one marking is located between the at least one row of loading apertures and the at least one row of collection apertures. In other embodiments the marking or markings are located between the electrophoresis lanes.

### BRIEF DESCRIPTION OF THE FIGURES

FIG. 1 is a schematic illustration of one embodiment of the electrophoresis cassette used in the methods disclosed herein.

FIGS. 2A and 2B are cross section schematic illustrations (taken along line IV of FIG. 1) of two embodiments of the electrophoresis cassettes used in the methods disclosed herein.

FIG. 3 is a schematic representation of a separation and isolation method with one collection well (CW) associated with a loading well (LW), wherein t refers to time, dotted lines indicate that some time has elapsed, * indicates a 1^{st} band of interest and ** indicates a 2^{nd} band of interest.

FIG. 4 is a schematic representation of another separation and isolation method with one collection well (CW) associated with a loading well (LW), wherein t refers to time, dotted lines indicate that some time has elapsed, * indicates a 1^{st} band of interest and ** indicates a 2^{nd} band of interest.

FIG. 5 is a schematic representation of a separation and isolation method with two collection wells (CW1 and CW2) associated with a loading well (LW), wherein the bands of interest are not resolved at CW1. Here t refers to time, dotted lines indicate that some time has elapsed, * indicates a 1^{st} band of interest and ** indicates a 2^{nd} band of interest.

FIG. 6 is a schematic representation of a separation and isolation method with three collection wells (CW1, CW2 and CW3) associated with a loading well (LW), wherein the bands of interest are not resolved at CW1. Here t refers to time, dotted lines indicate that some time has elapsed, * indicates a 1^{st} band of interest and * * indicates a 2^{nd} band of interest.

FIG. 7 is a schematic representation of a two-dimensional separation and isolation method with one collection well (CW) associated with a loading well (LW), wherein t refers to time, dotted lines indicate that some time has elapsed, and * indicates a 1^{st} band of interest.

FIG. 8 is a schematic representation of another two-dimensional separation and isolation method with one collection well (CW) associated with a loading well (LW), wherein t refers to time, dotted lines indicate that some time has elapsed, and * indicates a 1^{st} band of interest.

FIG. 9A-F are schematic representations of aperture and well patterns of the electrophoresis cassettes used in the methods disclosed herein.

FIG. 10A-E are schematic representations of aperture and well patterns of the electrophoresis cassettes used in the methods disclosed herein, wherein the relative positions of loading wells (LW) and collection wells(CW) is shown.

FIG. 11A-F are schematic representations of aperture and well patterns of the electrophoresis cassettes used in the two-dimensional separation and isolation methods disclosed herein.

FIG. 12A-E are schematic representations of aperture and well patterns of the electrophoresis cassettes used in the two-dimensional separation and isolation methods disclosed herein, wherein the relative positions of loading wells (LW) and collection wells(CW) is shown.

FIG. 13A-C show the images taken for the isolation and collection of the 800 bp (fourth band) of interest. FIG. 13A is an image of the first band (100 bp), second band (200 bp) and third band (400 bp) after passing through the collection well. FIG. 13B is an image of the fourth band entering the collection well. FIG. 13C is an image of the 800 bp fourth band run on another E-GEL® cassette (2% double comb containing ethidium bromide).

FIG. 14A-C show the images taken for the isolation and collection of a pre-stained ˜21 kD protein marker. FIG. 14A is an image of the ˜21 kD protein marker (pink band) at the edge of the collection well. FIG. 14B is an image of the ˜21 kD protein marker in the collection well. FIG. 14C is an image of the ˜21 kD protein marker T run on different gel.

A better understanding of the features and advantages of the present methods and compositions may be obtained by reference to the following detailed description that sets forth illustrative embodiments, in which the principles of our methods, compositions, devices and apparatuses are utilized, and the accompanying drawings.

### DETAILED DESCRIPTION OF THE INVENTION

Disclosed herein are electrophoresis systems, assemblies, apparatuses, cassettes and methods that can be used to separate, isolate and collect sample components from an electrophoresis gel, contained within such electrophoresis cassettes, after separating the sample components using electrophoresis. The sample component, which can be separated, isolated and collected using the methods disclosed herein, can be any molecule which can be separated by electrophoresis. Such molecules include, but are not limited to biomolecules such as peptides, polypeptides, proteins, oligonucleotides, DNA and RNA. These electrophoresis systems, assemblies, apparatuses, cassettes and methods provide a simpler and more efficient method than previous methods for isolating biomolecules in a gel-separated band. The electrophoresis systems, assemblies, apparatuses, cassettes and methods are especially well-suited for disposable commercial products.

The electrophoresis systems, assemblies, cassettes and methods disclosed herein can be used to separate, isolate and collect any molecule that can be separated from other molecules using gel electrophoresis. The electrophoretic separation can be based on differences in electrophoretic mobility (i.e. charge), molecular weight (i.e. mass/charge ratio) or combinations thereof, for example. By way of example only, the electrophoresis systems, assemblies, apparatuses, cassettes and methods disclosed herein can be used to separate, isolate and collect nucleic acids, such as oligonucleotides, DNA, and RNA, as well as peptides, polypeptides, and proteins. In addition, the electrophoresis systems, assemblies, apparatuses, cassettes and methods disclosed herein can be used to further purify nucleic acids, such as oligonucleotides, DNA, and RNA, as well as peptides, polypeptides, and proteins from other charged (positively charged or negatively charged) or uncharged (neutral) material which may or may not be a contaminant.

Definitions

Unless defined otherwise, all technical and scientific terms used herein have the same meaning as commonly understood by one of ordinary skill in the art to which this invention belongs. Generally, the nomenclature used herein are well known and commonly employed in the art. Terms of orientation such as “up” and “down”, “top” and “bottom”, “above” and “underneath” or “upper” or “lower” and the like refer to orientation of parts during use of a device. Where a term is provided in the singular, the inventors also contemplate the plural of that term. Where there are discrepancies in terms and definitions used in references that are incorporated by reference, the terms used in this application shall have the definitions given herein. As employed throughout the disclosure, the following terms, unless otherwise indicated, shall be understood to have the following meanings.

The term “sample”, as used herein, refers to a mixture of a plurality of unique molecular species which can be separated using gel electrophoresis. By way of example only, a sample may be a mixture of nucleic acids, a mixture of oligonucleotides, a mixture of DNA, a mixture of RNA, or combinations thereof. In addition, by way of example only, a sample may be a mixture of amino acids, a mixture of peptides, a mixture of proteins, or combinations thereof. Also, by way of example only, a sample may be a mixture of a molecular species and a plurality of contaminants.

The term “ambient temperature” as used herein, refers to the temperature in the range of 20° C. to 25° C.

As used herein, a biopolymer or biomolecule includes, but is not limited to, a nucleic acid, a protein, a polysaccharide, a lipid, and other macromolecules. A nucleic acid includes DNA, RNA, oligonucleotides, and fragments and analogs thereof. Nucleic acid sequences may be derived from genomic DNA, RNA, mitochondrial nucleic acid, chloroplast nucleic acid and other organelles with separate genetic material.

As used herein, proteins are complex, three-dimensional substances comprising one or more long, folded polypeptide chains. These chains, in turn, include of small chemical units called amino acids. All amino acids contain carbon, hydrogen, oxygen, and nitrogen. Some also contain sulfur. A “peptide” is a compound that includes two or more amino acids. The amino acids link together in a line to form a peptide chain. There are 20 different naturally occurring amino acids involved in the biological production of peptides, and any number of them can be linked in any order to form a peptide chain. The naturally occurring amino acids employed in the biological production of peptides all have the L-configuration. Synthetic peptides can be prepared employing conventional synthetic methods, using L-amino acids, D-amino acids or various combinations of amino acids of the two different configurations. Some peptide chains contain only a few amino acid units. Short peptide chains, e.g., having less than ten amino acid units, are sometimes referred to as “oligopeptides”, where the prefix “oligo” signifies “few.” Other peptide chains contain a large number of amino acid units, e.g., up to 100 or more, and are referred to a “polypeptides”, where the prefix “poly” signifies “many.” Still other peptide chains, containing a fixed number of amino acid units are referred to using a prefix that signifies the fixed number of units in the chain, e.g., an octapeptide, where the prefix “octa” signifies eight. (By convention, a “polypeptide” can be considered as any peptide chain containing three or more amino acids, whereas an “oligopeptide” is usually considered as a particular type of “short” polypeptide chain. Thus, as used herein, it is understood that any reference to a “polypeptide” also includes an oligopeptide. Further, any reference to a “peptide” includes polypeptides, oligopeptides. Each different arrangement of amino acids forms a different polypeptide chain. In certain non-limiting examples, the polypeptide includes between 40 and 4000 amino acids, between 50 and 3000 amino acids, or between 75 and 2000 amino acids.

As used herein, a “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogues thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be disclosed herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation. (see Sambrook et al. Molecular Cloning, A Laboratory Manual, Cold Spring Harbor Laboratory Press).

The terms “aligned” or “alignment”, as used herein, refers to at least two objects which are arranged in a line. As used herein objects can be aligned or in alignment when their respective centers are shifted from this line by up to 50% of the width of the object.

The term “chaotropic agent” or “chaotrope”, as used herein, refers to any substance capable of altering the secondary and tertiary structure of proteins and nucleic acids.

The term “contaminant” as used herein, refers to any component of the sample that is not isolated and collected using the methods and apparatuses described herein. By way of example only, contaminants include, but are not limited to, salts, buffers, proteins, peptides, nucleic acids and amino acids.

The term “electrophoresis lane”, as used herein, refers to the area between a loading well and its corresponding collection well. The width of the loading well or collection well (whichever is larger) will define the width of the electrophoresis lane.

Methods for Isolating Biomolecules and/or Sample Bands

Reference will now be made in detail to the embodiments of the electrophoresis systems, assemblies, apparatuses, cassettes and methods which can be used to separate, isolate and collect sample components from an electrophoresis gel, examples of which are illustrated in the accompanying figures.

The methods disclosed herein can be a two-well method in which there is one loading well and one collection well, or the methods can be multi-well methods, in which the methods include an electrophoresis gel with more than one loading well and more than one collection well. Various embodiments of the two-well method and multi-well methods are presented herein.

In one aspect provided herein is a method for isolating a biomolecule, or sample band from an electrophoresis gel that includes the following:

(1) providing or obtaining a closed electrophoresis cassette wherein the electrophoresis cassette includes the following: (i) a separation chamber having walls, in which at least one of the walls has at least one row of loading apertures and at least one row of collection apertures; (ii) an electrophoresis gel matrix contained within the separation chamber; wherein the electrophoresis gel matrix has at least one row of loading wells and at least one row of collection wells therein, with each loading well accessible through a loading aperture and each collection well accessible through a collection aperture, and each loading well is aligned with at least one collection well in an electrophoresis lane; and (iv) two electrodes, wherein the rows of wells and apertures are located between the electrodes.

(2) loading a sample that contains a plurality of components, including a biomolecule (such as a protein) of interest, into at least one loading well through at least one loading aperture of the electrophoresis cassette.

(3) applying an electric field between the two electrodes to drive electrophoretic migration of the biomolecule into a collection well, and

(4) removing the liquid containing the biomolecule or band of interest from the collection well through the collection aperture, thereby isolating the biomolecule or band of interest.

A loading well is typically accessible through a loading aperture by being located underneath the loading aperture such that the tip of a microliter-scale liquid handling instrument such as a micropipettor can pass through the aperture and dispense liquid into the well. A collection well is typically accessible through a collection aperture by being located underneath the collection aperture such that the tip of a microliter-scale liquid handling instrument such as a micropipettor can pass through the aperture and remove liquid from the well,

During methods of the invention, when the electric field is applied, a liquid is present in the collection well. Therefore, the method typically includes filling the collection wells with a liquid, such as water or buffer, through the collection aperture

Non-limiting examples of electrophoresis gel cassettes used in the methods disclosed herein, and non-limiting examples of the apparatuses in which such cassettes can be used in, have been disclosed in U.S. Pat. No. 5,582,702, U.S. Pat. No. 5,865,974, and U.S. Pat. No. 6,562,213, each of which is herein incorporated by reference in its entirety.

In certain illustrative embodiments, the electrophoresis cassettes used in the methods disclosed herein are closed electrophoresis cassettes. Closed electrophoresis gel cassette have at least four walls which are sealed to form a separation chamber surrounded by the walls, and the separation chamber contains an electrophoresis gel matrix which has at least two (2) wells formed therein. In addition, at least one wall of such cassettes has an array of openings (also referred to herein as apertures) for access from outside of the cassette to the wells formed in the electrophoresis gel contained in the separation chamber, or for access from outside of the cassette to the empty separation chamber inside the cassette. Such closed cassettes also include all the chemical compounds required for driving electrophoresis separations and, in certain embodiments, for enabling visualization of the separated sample bands. In addition, such closed cassettes can be disposable. In certain embodiments the electrophoresis gel is cast/polymerized in the separation chamber by an outside vendor, while in other embodiment the separation chamber does not contain an electrophoresis gel until an end user casts/polymerizes the electrophoresis gel into the separation chamber before using the electrophoresis cassette.

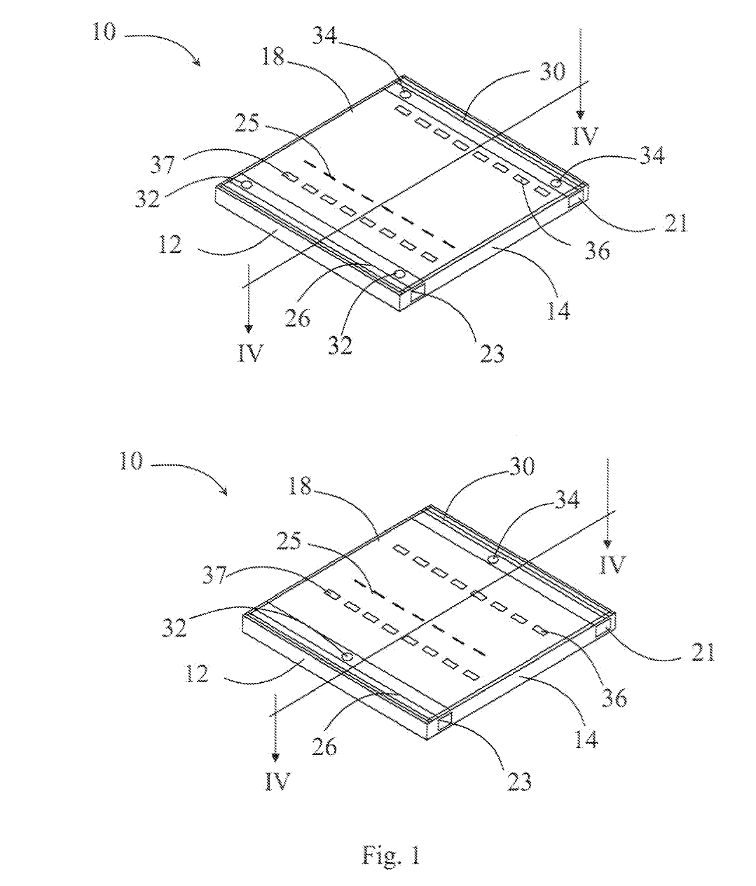

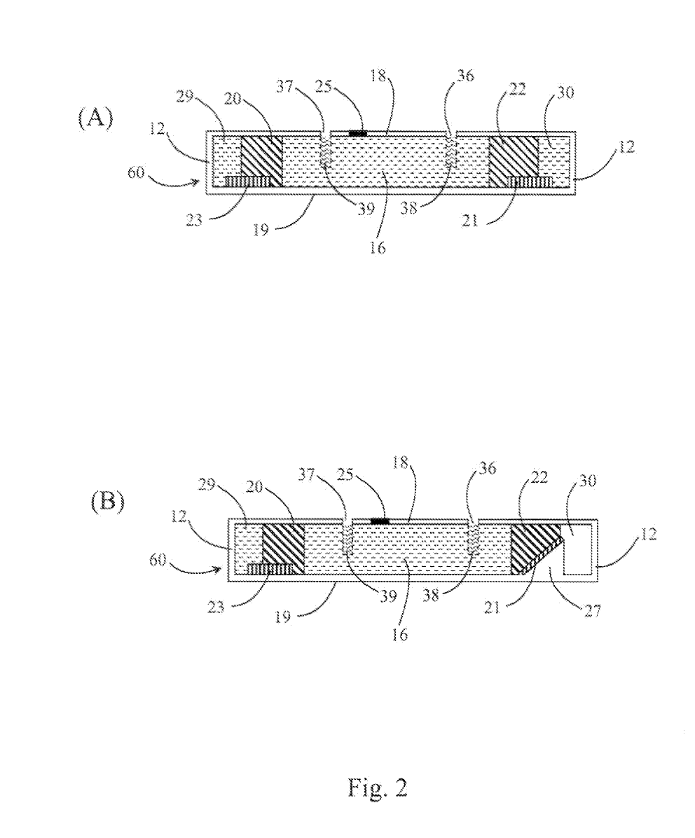

FIGS. 1 and 2 illustrate one embodiment of the closed cassettes used in the methods disclosed herein. FIG. 1 is an external view, wherein FIG. 2 is seen in the cross section illustration along IV-IV of FIG. 1. Cassette 10 comprises a three dimensional separation chamber having a bottom wall 19, side walls 12 and 14, and a top wall 18 each of which has a specified thickness. Cassette 10 is substantially closed in that it is enclosed by walls 12, 14, 16, and 19, but it also comprises apertures (36 and 37) as will be disclosed herein, and can also comprise optional vent holes (34 and/or 32). The thickness of the walls can range from 0.1-10 mm, and in certain embodiments the thickness is 1.5 mm. In other embodiments the thickness is 9 mm, 8 mm, 7 mm, 6 mm, 5 mm, 4 mm, 3 mm, 2 mm or 1 mm.

The length of cassette 10 can range from 50 to 200 mm, the width of cassette 10 can range from 50-150 mm and the height of cassette 10 can range from 1-10 mm. In one embodiment, the length, width and height of cassette 10 are 160 millimeters (mm), 100 mm and 6 mm, respectively. In another embodiment, the length, width and height of cassette 10 are 130 mm, 130 mm and 6 mm, respectively. In another embodiment, the length, width and height of cassette 10 are 100 mm, 80 mm and 6 mm, respectively. In another embodiment, the length, width and height of cassette 10 are 108 mm, 135 mm and 6.7 mm, respectively.

Cassette 10 may optionally include vent holes 32 and 34 to allow escape of gaseous molecules (e.g., oxygen and/or hydrogen) that might be generated during electrophoresis, due to the electrochemical reaction at electrodes 23 and 21. In certain embodiments the vent holes range in diameter from 0.5-2 mm, while in other embodiments the vent holes range in diameter from 0.5-1 mm. In other embodiments the vent holes range in diameter from 1-2 mm, and in one embodiment, the vent holes are 1 mm in diameter. The number of vent holes associated with each electrode can be from 1-10. In certain embodiments, the number of vent holes associated with each electrode can be from 1-6, while in other embodiments the number of vent holes associated with each electrode can be from 1-3. Still, in other embodiments, the number of vent holes associated with each electrode can be from 1-2, while in other embodiments the one vent hole is associated with each electrode. The vent hole or holes may be positioned anywhere above the electrode, and FIG. 1 shows two possible embodiments for the positions of vent holes included in the cassettes used with the methods disclosed herein.

In certain embodiments apertures 36 are loading apertures and apertures 37 are collection apertures, while in other embodiments apertures 36 are collection apertures and apertures 37 are loading apertures. Cassette 10 may include markings 25 which are located a specified distance from the apertures 36 and 37. In one embodiment, shown in FIGS. 1 and 2, markings 25 are located a specified distance from the collection apertures 37, however such markings can also be located a specified distance from the loading apertures 36. In a non-limiting embodiment shown in FIGS. 1 and 2, the markings 25 are located between apertures 36 and 37 with the markings 25 located along a line parallel to the apertures 36 and 37, but not in direct alignment with apertures 36 and 37 (as shown in FIG. 1). In alternative embodiments the markings 25 are located between apertures 36 and 37 and directly aligned with the apertures 36 and 37. In other embodiments markings 25 can be located between apertures 36 and electrode 21, and in other embodiments markings 25 can be located between apertures 37 and electrode 23. In further embodiments markings 25 can be located anywhere between electrodes 21 and 23. The function of such markings is disclosed herein. The distance the markings are from the apertures can range from 1-100 mm. In certain embodiments the distance ranges from 1-75 mm, while in other embodiments the distance ranges from 1-50 mm. In other embodiments the distance ranges from 1-25 mm, and in other embodiments the distance ranges from 1-10 mm. In other embodiments the distance ranges from 1-5 mm, and in one embodiment the distance is 1 mm.

As seen in the cross section illustration 60 of FIGS. 2A and 2B, the separation chamber comprises an electrophoresis gel matrix 16, which may be any suitable electrophoresis gel matrix and will be discussed below. The separation chamber also comprises two conductive electrodes referenced 21 and 23 which, when connected to an external direct current (DC) electrical power source, provide the electric field required to drive electrophoresis separation. In the illustrated embodiment in FIGS. 1 and 2, electrode 21 is the cathode and electrode 23 is the anode, however in other embodiments electrode 21 is the anode and electrode 23 is the cathode.

The separation chamber may optionally comprise ion exchange matrices, referenced 20 and 22. In the illustrated embodiment in FIGS. 1-3, electrode 21 is the cathode, electrode 23 is the anode, matrix 20 is a cation exchange, and matrix 22 is an anion exchange matrix, however in other embodiments electrode 21 is the anode, electrode 23 is the cathode, matrix 20 is an anion exchange, and matrix 22 is a cation exchange matrix.

The separation chamber may also optionally comprise internal volumes 29 and 30 which, independent of each other, may be unoccupied, occupied with electrophoresis gel matrix, or occupied with buffer. If unoccupied the internal volume 29 or 30 is used to as a volume in which gases produced during electrophoresis may accumulate. Alternatively, as noted above, cartridge 10 may optionally include at least two vent holes 32 and 34 (one hole for each electrode) for venting the gases accumulated in the volumes 29 and/or 30. It will be appreciated that if the cassette 10 includes vent holes 32 and 34 they are opened just before the electrophoresis begins and are closed after the test is completed to reduce the possibility of contamination.

In addition, cartridge 10 may comprise a ramp 27 which can support electrode 21. The ramp facilitates continuous contact between electrode 21 and the surface of the ion exchange matrix 22 overlying electrode 21, whereby release of gas bubbles produced at the vicinity of electrode 21 are directed towards empty volume 30. In certain embodiments, the ramp 27 is formed as an integral part of cartridge 10 and is inclined to the bottom wall 19 at an angle of about 45 degrees.

Also shown in the cross section illustration 60 of FIGS. 2A and 2B, are wells 38 and 39 which are formed in electrophoresis gel matrix 16. Such wells are located underneath apertures 36 and 37. As shown in FIG. 1 apertures 36 and 37 and the corresponding wells 38 and 39 are configured in two rows, one row used to introduce samples of biomolecules which are to undergo electrophoretic separation and the other row to collect a biomolecule or population of molecules of interest. However, the configuration of the apertures and wells used in the methods disclosed herein are not limited to two rows and can include other configurations, such as various types of arrays, as discussed herein. In one embodiment, cartridge 10 comprises a plurality of apertures and a plurality of wells, wherein the plurality of apertures and plurality of wells range from 1-200 apertures and 1-200 wells. In another embodiment, the plurality of apertures and plurality of wells range from 1-100 apertures and 1-100 wells. In another embodiment, the plurality of apertures and plurality of wells range from 1-50 apertures and 1-50 wells. In certain embodiments, cartridge 10 comprises 96 apertures and 96 wells, while in certain embodiments, cartridge 10 comprises 48 apertures and 48 wells. The dimensions of apertures 36 and 37 and wells 38 and 39 are discussed herein, however in one embodiments wells 38 and 39 have dimensions of 0.5-5 mm wide, 1-5 mm long, and 3-5 mm deep.

The wells may be formed by any suitable method, such as by introducing a “comb” into the electrophoresis gel matrix within the separation chamber during the assembly of the electrophoresis gel matrix when the electrophoresis gel matrix is still in a liquid state. The “comb” has protruding teeth positioned so that the teeth project into the electrophoresis gel matrix via the apertures in the top wall 18. The wells form in the electrophoresis gel matrix when the matrix solidifies into a gel state around the comb features. When the comb is pulled out of the electrophoresis gel matrix and the apertures the wells are available for loading with liquid, such as sample, buffer, or water. The comb may be removed just before loading, or it may be removed some time before loading, such as in the range of from 5 seconds to 1 day before loading, or 10 seconds to 12 hours before loading, or 10 seconds to 30 minutes before loading. Alternatively, the comb is pulled out of the electrophoresis gel matrix and the apertures and the top of the cassette (including the apertures and corresponding wells) is covered with tape thereby sealing the resulting wells from potential contamination. The sealing tape is then removed just before loading, or it may be removed some time before loading, such as in the range of from 5 seconds to 1 day before loading, or 10 seconds to 12 hours before loading.

In the methods for the separation, isolation and collection of a biomolecule of interest disclosed herein, the apertures 36 and 37 and wells 38 and 39 can be designated as either loading or collecting apertures or wells. Loading apertures, with their corresponding loading wells located underneath, are used as to load sample into the loading wells, while collection apertures are used to collect the biomolecule of interest from collection well located underneath the collection apertures.

With reference to FIGS. 1 and 2 the method for the separation, isolation and collection of a biomolecule of interest disclosed herein are as follows: prior to electrophoretic separation, samples of biomolecules are placed into the loading wells 38 via the loading apertures 36, while liquid, such as buffer or water, is placed into the collection wells 39 via the collection apertures 37. Any unused loading wells can also be filled with liquid. An external electrical power source connected to electrodes 21 and 23 is turned on to provide the electric field required to drive electrophoretic migration of the sample components and drive electrophoretic separation of the components into discrete bands. Each band either comprises a single biomolecular species or it can comprise a mixture of species with similar electrophoretic migration characteristics. The electrophoretic separation is allowed to continue until a sample band of interest, comprising a biomolecule of interest, enters collection wells 39, wherein the electrical power source is turned off. The liquid inside collection wells 39 is then removed through collection apertures 37. The liquid removed from collection wells 39 contains the sample band of interest which comprises the biomolecule of interest. The collected sample band can then be used for analytical evaluation or it can be used in biological processes, such as cloning. In certain embodiments the biochemical process is restriction enzyme cloning, while in other embodiments the biochemical process is high-throughput recombination cloning. In certain embodiments the biochemical process is TOPO® (Invitrogen Corp., Carlsbad) restriction cloning, while in other embodiments the biochemical process is GATEWAY® (Invitrogen Corp., Carlsbad) recombination cloning. In certain embodiments, the efficiencies of such cloning methods are enhanced 10-1000 fold, while in other embodiments the efficiency is enhanced 10-500 fold. In certain embodiments, the efficiencies of such cloning methods are enhanced 10-100 fold. In addition, if necessary, the collected sample can be further purified by repeating the method disclosed herein. The electrical power source is typically a direct current (DC) power source.

In other embodiments the collection well 39 can be refilled with liquid and the direct current (DC) electrical power source is turned on again to continue the migration and separation of the bands remaining in the electrophoresis gel matrix 16. The electrophoretic separation is allowed to continue until a different sample band of interest, comprising a different biomolecule of interest, enters collection wells 39, wherein the direct current (DC) electrical power source is again turned off. The liquid inside collection wells 39 is then removed through collection apertures 37 and the collected sample band can be used as disclosed above.

In other embodiments the collection well 39 can be refilled with liquid and the direct current (DC) electrical power source is turned on again, but the polarity is reversed. The migration and separation of the bands remaining in the electrophoresis gel matrix 16 continues but in the reverse direction. The electrophoretic separation is allowed to continue until a different sample band of interest, which had passed through collection wells 39 during a previous separation run, reenters collection wells 39 and the direct current (DC) electrical power source is again turned off. The liquid inside collection wells 39 is then removed through collection apertures 37 and the collected sample band can be used as disclosed above.

In other embodiments the reverse mode disclosed above can be applied if the band of interest, or portion thereof, has migrated through the collection well prior to terminating the electric field. In such embodiments the electric field is reversed and the missed band, or portions thereof, migrates back into the collection well, wherein it can be collected as disclosed above.

In other embodiments the reverse mode disclosed above can be used to improve the collection yield of diffuse bands which have a band width larger than the collection well width. In such embodiments the diffuse band of interest is allowed to migrate through the collection well until the trailing end enters the collection well, wherein the electric field is terminated and the liquid in the collection well is removed. The collection well is then refilled with liquid and the electric field is reapplied with the polarity reversed. The remaining portion of the band of interest migrates back into the collection well and is collected as disclosed above. Although these embodiments use two fractions to collect and isolate a band of interest it is understood that the number of fractions used to collect and isolate a band of interest can also be 3, 4, 5, 6, 7, 8, 9, 10 or more fractions. In certain embodiments the number of fractions used depends on the width of the migrating band relative to the collection well width. The wider the band the more fractions may be needed.

In other embodiments, the collection yield for diffuse bands does not use the reverse mode. In such embodiments the diffuse band of interest is allowed to migrate into the collection well until the leading end is about to exit the collection well, wherein the electric field is terminated and the liquid in the collection well is removed. The collection well is then refilled with liquid and the electric field is reapplied. The remaining portion of the band of interest migrates into the collection well and is collected as disclosed above. Although these embodiments use two fractions to collect and isolate a band of interest it is understood that the number of fractions used to collect and isolate a band of interest can also be 3, 4, 5, 6, 7, 8, 9, 10 or more fractions. In certain embodiments the number of fractions used depends on the width of the migrating band relative to the collection well width. The wider the band the more fractions may be needed.

In alternative embodiments, markings 25 can be used to calculate the time the band of interest will enter collection wells 39 by dividing the distance markings 25 are from the collection aperture 37 by the electrophoretic migration rate of the sample band.

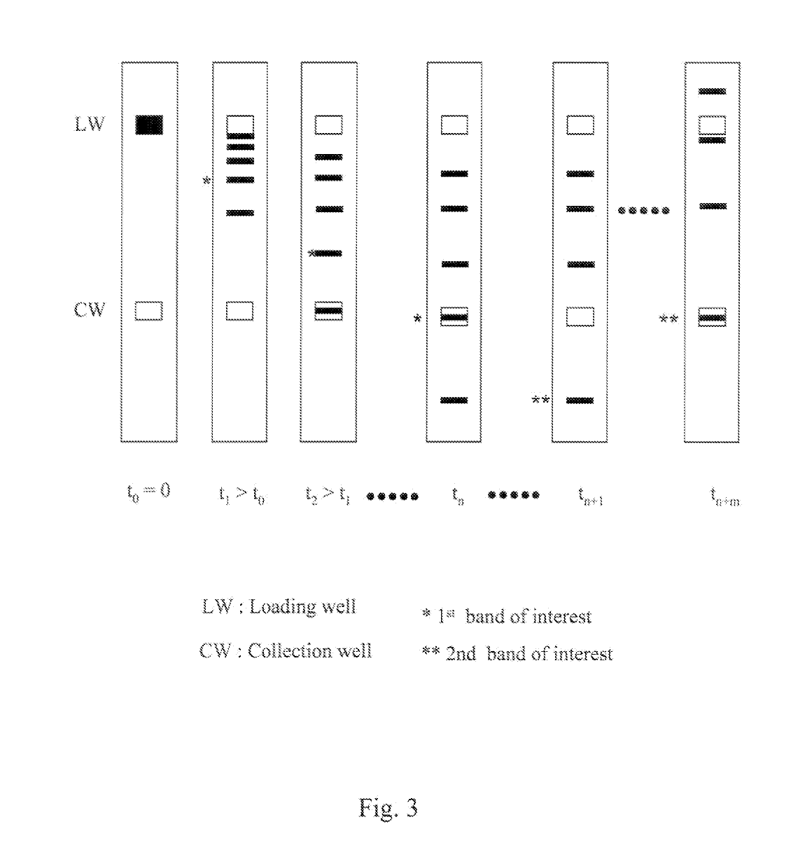

FIGS. 3-6 illustrate non-limiting embodiments disclosed herein of the methods for the isolation of a biomolecule and/or a band or bands from an electrophoresis gel. A two-well method is shown in FIG. 3 where a sample is loaded through a loading aperture in an electrophoresis cassette into a loading well (LW) located within the electrophoresis gel enclosed in the electrophoresis cassette. The sample is believed to contain, and typically contains at least two unique molecular species to be electrophoretically separated, wherein at least one of the unique molecular species is to be isolated from the electrophoresis gel after the sample components have been separated. Prior to the application of an electric field (t=0) the sample is located in a loading well in the electrophoresis gel and water or buffer is loaded into a collection well (CW). An electric field is then applied and the sample components begin migrating toward the collection well. After some time (t₁>t₀) the sample components separate into discrete bands as they continue to migrate toward the collection well. The electrophoretic separation and migration of the sample components is continued until a sample component band of interest (as shown by the *) migrates into the collection well at time (tₙ), whereupon the electric field is terminated. Note that in FIGS. 3-6 the large dotted line indicates the progression of time. The sample component within the collection well is removed and is either further analyzed using various applicable analytical techniques or is used in a chemical, biochemical or molecular biological process.

Any sample component band migrating ahead of the component band of interest will migrate through the collection well, back into the electrophoresis gel and continue migrating in the gel. Also shown in FIG. 3 is the embodiment wherein, if such components are of interest after the initial removal of the first band of interest, then the collection well is refilled with water or buffer and the polarity of the electric field is reversed to cause such bands to migrate back toward the collection well (tₙ₋₁). When the second band of interest is within the collection well the electric field is again terminated and the second band of interest is removed from the collection well at time (tₙ₊ₘ).

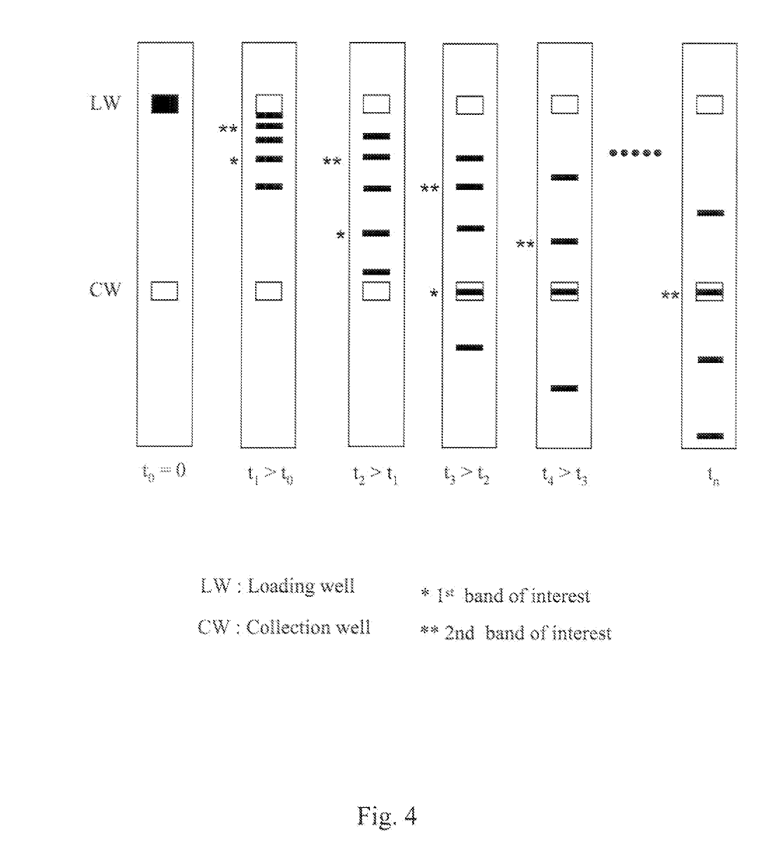

Another embodiment of the two-well method is shown in FIG. 4, wherein a sample is loaded into a loading well located within the electrophoresis gel and at least two sample bands of interest are to be removed from the electrophoresis gel after the sample components have been separated. Prior to the application of an electric field (t=0) the sample is located in a loading well in the electrophoresis gel and water or buffer is loaded into a collection well (CW). An electric field is then applied and the sample components begin migrating toward the collection well. After some time (t₁>t₀) the sample components separate into discrete bands as they continue to migrate toward the collection well. The electrophoretic separation and migration of the sample components is continued until the first sample component band of interest (as shown by the *) migrates into the collection well (t₃>t₂), whereupon the electric field is terminated. The sample component within the collection well is removed and is either further analyzed using various applicable analytical techniques or is used in a chemical, biochemical or molecular biological process. The collection well is then refilled with water or buffer and the electric field is reapplied until the second band of interest migrates into the collection well (tₙ), whereupon the electric field is terminated and the second sample component is removed from the collection well. The second sample component may be further analyzed using various applicable analytical techniques or is used in a chemical, biochemical or molecular biological process. Again any sample bands which migrated ahead of the bands of interest can be isolated and removed from the electrophoresis gel by reversing the polarity as disclosed above.

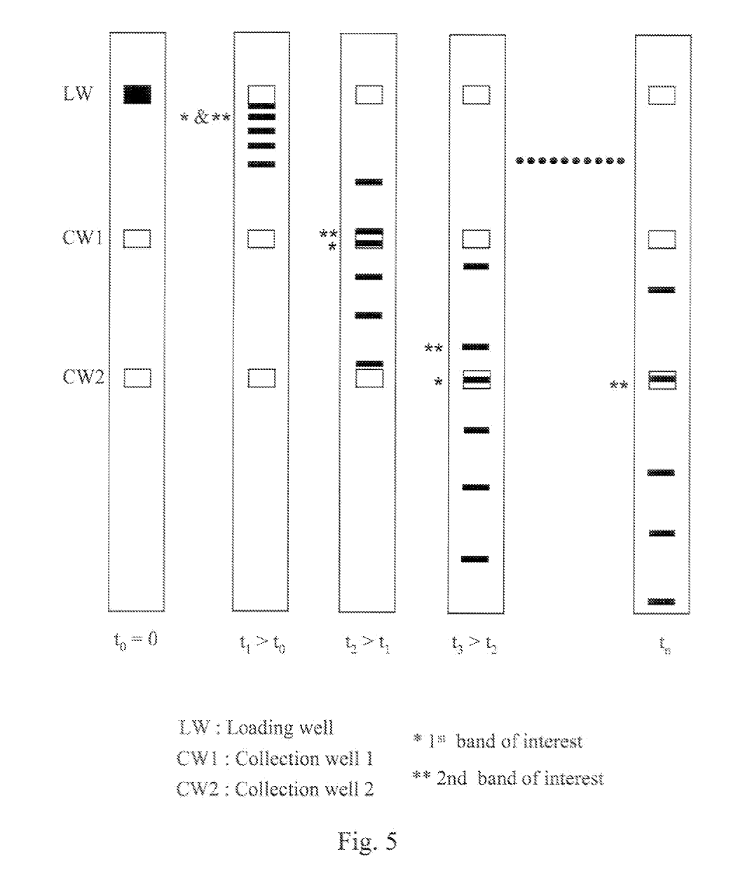

FIG. 5 illustrates one non-limiting embodiment of a multi-well method to separate, isolate, and collect at least two sample components of interest, wherein the sample components have not resolved before migrating into the collection well. The multi-well method disclosed in this embodiment uses an electrophoresis gel having one loading well (LW) and two collection wells (CW1 & CW2) aligned with each other in an electrophoresis lane. In this embodiment, prior to the application of an electric field (t=0), a sample is loaded into the loading well located within the electrophoresis gel and water or buffer are loaded into the collection wells (CW1 & CW2). An electric field is then applied and the sample components begin migrating toward the collection wells. After some time (t₁>t₀) most of the sample components have separated into discrete bands, however the two components of interest remain unresolved. The sample components continue to migrate toward the first collection well (CW1), and when the two components of interest enter CW1 (t₂>t₁) they remain unresolved. The electrophoretic separation and migration of the sample components is continued until the first band of interest (as shown by the *) and the second band of interest (shown as **) resolve and the first band of interest migrates into the second collection well (CW2) at time (t₃>t₂). The electric field is terminated and the first sample component is removed from CW2 and is either further analyzed using various applicable analytical techniques or is used in a chemical, biochemical or molecular biological process. The collection well is then refilled with water or buffer and the electric field is reapplied until the second band of interest migrates into CW2 at time (tₙ), whereupon the electric field is terminated and the second sample component is removed from the collection well. The second sample component may be further analyzed using various applicable analytical techniques or is used in a chemical, biochemical or molecular biological process. Again any sample bands which migrated ahead of the bands of interest can be isolated and removed from the electrophoresis gel by reversing the polarity as disclosed above.

It will be understood that the method disclosed above and illustrated in FIG. 5 could be applied to the isolation of more than two components of interest, including components which have resolved prior to reaching CW1. In addition, it will be understood that difficult to resolve sample components can be separated, isolated and collected using a two-well method, rather than the multi-well method disclosed above, wherein the distance between the two wells (the loading well and the collection well) is increased to allow for the sample components to resolve. Alternatively, the gel composition and/or electric field may be altered to affect the separation characteristics of the sample components, and thereby allow for sufficient resolution of the sample bands and collection of the band or bands of interest.

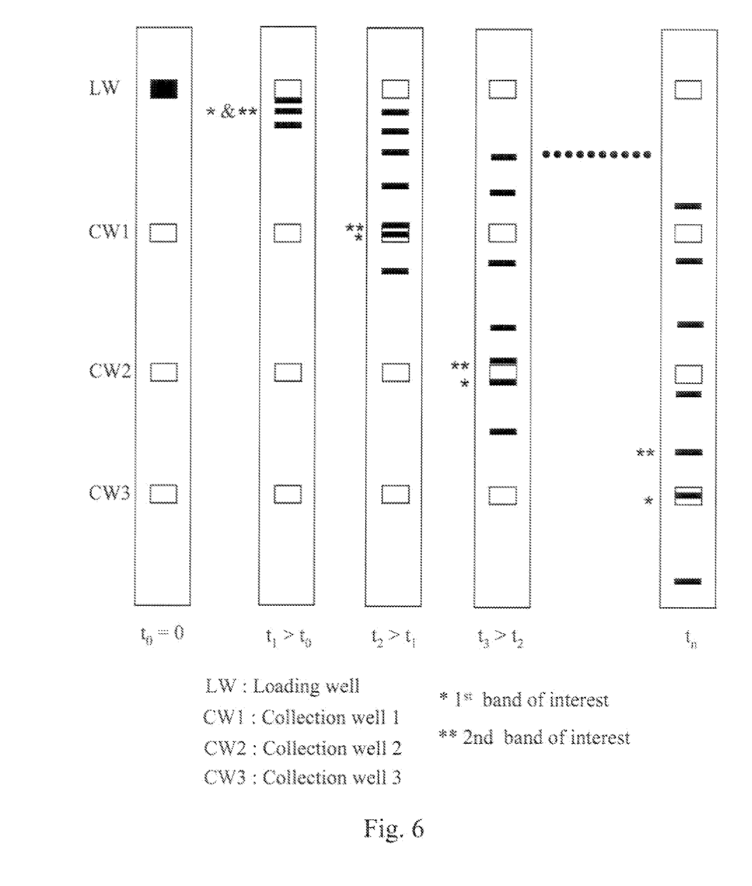

If the sample components of interest have not resolved before entering CW2, then another embodiment of a multi-well method which includes a third collection well (CW3) is illustrated in the FIG. 6. The two sample components of interest as shown in FIG. 6 can be separated, isolated and collected from the electrophoresis gel by using the method disclosed above.

It will be understood that if the two sample components of interest have not resolved before entering CW3, then the electrophoretic separation of the sample components of interest can be continued, wherein a fourth, fifth, sixth, seventh, eighth, and so on, collection well can be used until the bands resolve. The number of collection wells required will depend on the respective electrophoretic characteristics of the sample components. Alternatively, the electrophoresis gel properties and the electric field parameters can be modified thereby achieving band resolution without requiring an excessive number of collection wells or a very large gel. The various arrangements of wells, electrophoretic gels, and electric field parameters used in the methods disclosed herein are disclosed herein.

In certain embodiments of such methods DNA or large fragments thereof can be purified from smaller oligonucleotides or nucleic acids, wherein during electrophoresis the DNA or large fragments thereof do not migrate into the electrophoresis gel from a loading well. The smaller oligonucleotides or nucleic acids will migrate into the electrophoresis gel leaving the DNA or large fragments in the loading well. The purified DNA or large fragments are then removed from the loading well for further analysis using various applicable analytical techniques or for use in chemical, biochemical or molecular biological processes.

In certain embodiments of such methods RNA or large fragments thereof can be purified from smaller oligonucleotides or nucleic acids, wherein during electrophoresis the RNA or large fragments thereof do not migrate into the electrophoresis gel from a loading well. The smaller oligonucleotides or nucleic acids will migrate into the electrophoresis gel leaving the RNA or large fragments in the loading well. The purified RNA or large fragments are then removed from the loading well for further analysis using various applicable analytical techniques or for use in chemical, biochemical or molecular biological processes.

In certain embodiments of such methods large proteins can be purified from smaller peptides or amino acids, wherein during electrophoresis the large proteins do not migrate into the electrophoresis gel from a loading well. The smaller peptides or amino acids will migrate into the electrophoresis gel leaving large proteins in the loading well. The purified proteins are then removed from the loading well for further analysis using various applicable analytical techniques or for use in chemical, biochemical or molecular biological processes.

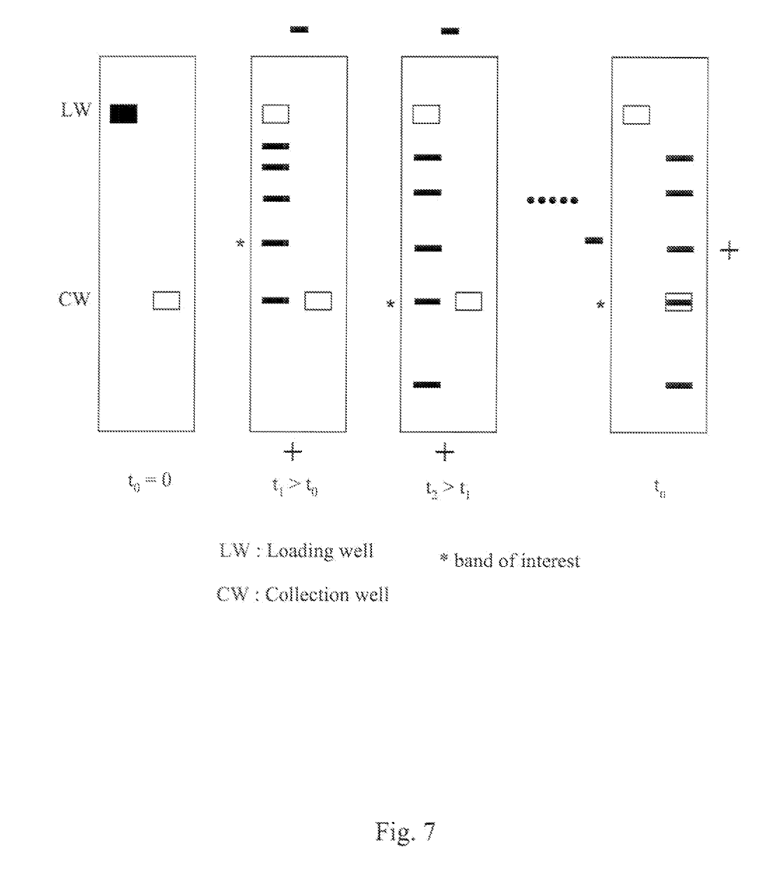

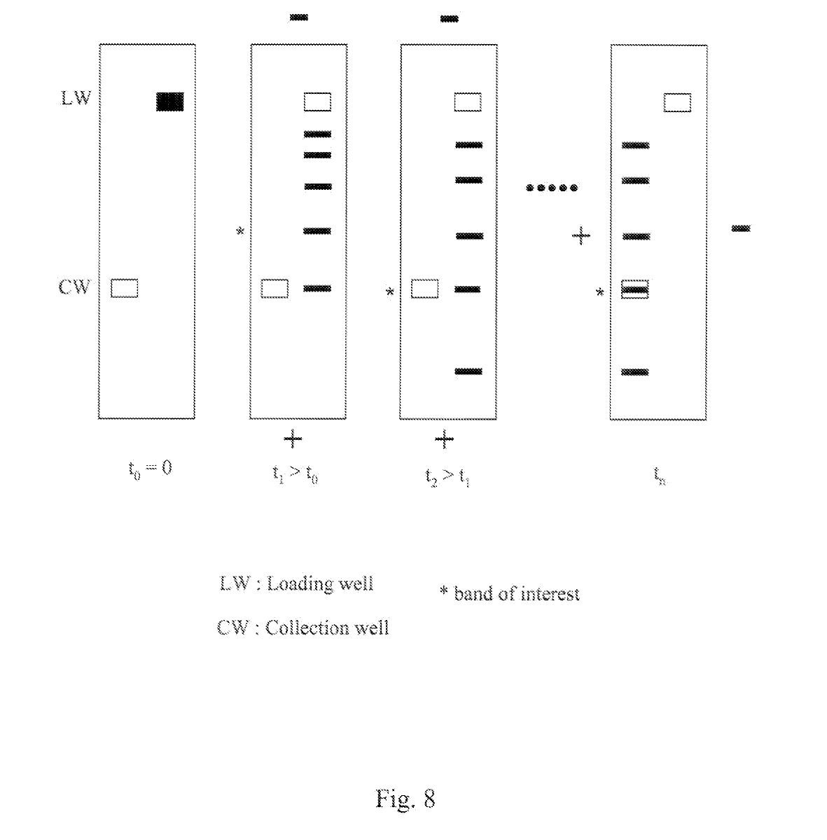

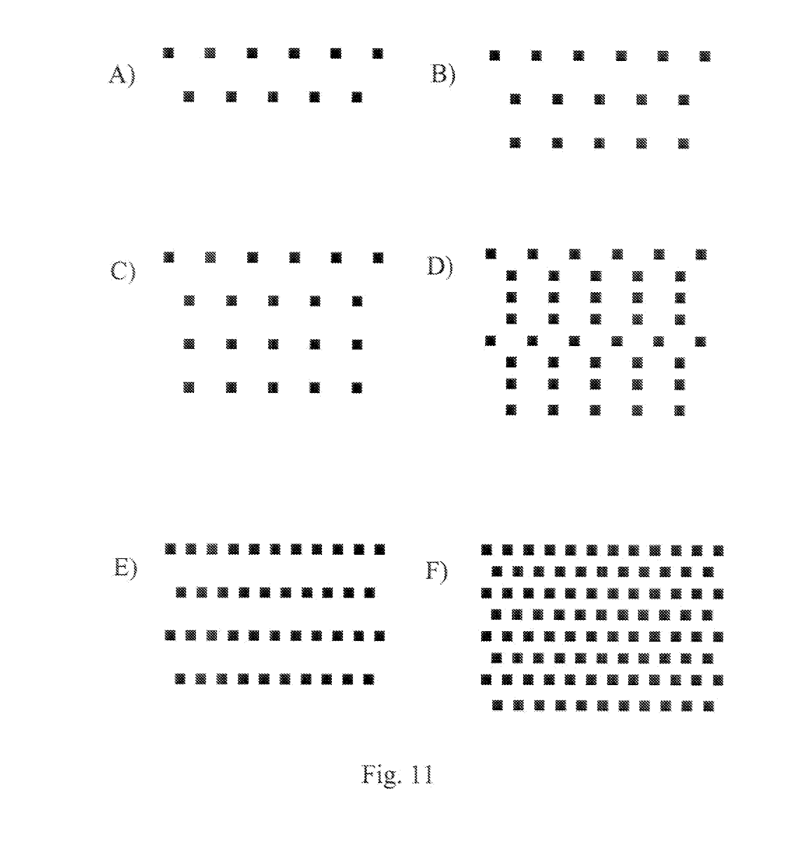

Another embodiment of the methods for separating, isolating and collecting a sample component of interest disclosed herein is shown in FIG. 7 and FIG. 8. In such methods the loading well is not in direct alignment with a collection well or collection well, instead the collection well or collection wells are in a parallel electrophoresis lane to the loading well (see FIG. 11) and two pairs of electrodes are used to separate, isolate and collect a band of interest by 2-dimensional (2D) electrophoresis. In such 2-dimensional (2D) electrophoretic methods a sample is loaded into a loading well (LW) located within the electrophoresis gel. The sample contains at least two unique molecular species to be electrophoretically separated, wherein at least one of the unique molecular species is to be isolated after the sample components have been separated. Prior to the application of the first electric field (t=0) the sample is located in a loading well in the electrophoresis gel and water or buffer is loaded into a collection well (CW). The first electric field is then applied between electrodes located above and below the loading well and collection well, respectively (as indicated by the + and − symbols in FIGS. 7 and 8). The sample components begin migrating in the direction of the collection well, but in a different electrophoresis lane than the collection well. After some time (t₁>t₀) the sample components separate into discrete bands as they continue to migrate. The electrophoretic separation and migration of the sample components is continued until a sample component band of interest (as shown by the *) is beside the collection well at time (t₂>t₁), whereupon the first electric field is terminated. The second electric field is then applied between electrodes located to the left and right of the loading and collection wells (i.e. orthogonal to the first pair of electrodes), wherein the separated sample bands migrate in the direction toward the collection well or wells until the sample component of interest is within the collection well. The sample component of interest is removed from the collection well and is either further analyzed using various applicable analytical techniques or is used in a chemical, biochemical or molecular biological process. FIG. 7 and FIG. 8 show the loading well at the top of the electrophoresis gel and the collection well at the bottom, however it would understood that the loading well can be at the bottom with the collection well at the top. In addition, it would be understood that such 2-D methods can be performed using multiple loading wells with multiple collection wells (see FIG. 11 for non-limiting examples of loading well and collection well configurations).

As will be understood, a biomolecule isolated according to the methods provided herein, or a sample component collected according to the methods provided herein, can be used or further analyzed by virtually any method known in the art for isolated biomolecules. For example, in certain embodiments the collected sample component of interest is further analyzed using mass spectrometry. For example, the collected sample component of interest can be used in a recombinant DNA procedure such as a cloning or ligation reaction. In certain embodiments the biochemical process is restriction enzyme cloning, while in other embodiments the biochemical process is high-throughput recombination cloning. In certain embodiments the biochemical process is TOPO® (Invitrogen Corp., Carlsbad) restriction cloning, while in other embodiments the biochemical process is GATEWAY® (Invitrogen Corp., Carlsbad) recombination cloning. In certain embodiments, the efficiencies of such cloning methods are enhanced 10-1000 fold, while in other embodiments the efficiency is enhanced 10-500 fold. In certain embodiments, the efficiencies of such cloning methods are enhanced 10-100 fold. As another example, the collected sample component of interest can be used in a PCR reaction, a fragment-length-polymorphism analysis, or a DNA sequencing reaction. In certain embodiments the collected sample component of interest is a protein that is subsequently used for the production of antibodies. In certain embodiments the collected sample component of interest is a purified oligonucleotide.

In certain embodiments a large quantity of the component of interest is obtained by pooling the volumes collected from an array of wells. In such embodiments an array of wells is used to purify the crude sample. In other embodiments, the volumes collected from a number of two well systems is pooled. In still other embodiments the sample is loaded into one large loading well and the sample component of interest is collected from one large collection well.

The percent recovery of the sample component of interest obtained using electrophoresis systems, assemblies, cassettes and methods disclosed herein can range from 10% up to 100%. In certain embodiments the percent recovery is from 50% to 95%, while in other embodiments the percent recovery is from 50% to 70%. In other embodiments the percent recovery is from 70% to 95%.

In the electrophoresis systems, assemblies, cassettes and methods disclosed herein can be used to separate, isolate and collect a nucleic acid sequence having from 10 bases to 10000 bases. In certain embodiments the electrophoresis systems, assemblies, cassettes and methods disclosed herein can be used to separate, isolate and collect a nucleic acid sequence having from 50 bases to 5000 bases. In certain embodiments the electrophoresis systems, assemblies, cassettes and methods disclosed herein can be used to separate, isolate and collect a nucleic acid sequence having from 100 bases to 5000 bases. In certain embodiments the electrophoresis systems, assemblies, cassettes and methods disclosed herein can be used to separate, isolate and collect a nucleic acid sequence having from 100 bases to 1000 bases.

In another aspect provided herein is a method for isolating a biomolecule or a sample band from an electrophoresis gel that includes the following:

(1) providing or obtaining an electrophoresis cassette wherein the electrophoresis cassette includes the following: (i) a separation chamber having walls, in which at least one of the walls has an array of apertures, including at least one row of loading apertures and at least one row of collection apertures; (ii) an electrophoresis gel matrix contained within the separation chamber; wherein the electrophoresis gel matrix has an array of wells, including at least one row of loading wells and at least one row of collection wells therein, with each loading well accessible through a loading aperture and each collection well accessible through a collection aperture, and each loading well is aligned with at least one collection well in an electrophoresis lane; and (iv) at least two electrodes, which include at least one anode and at least one cathode, wherein the rows of wells and apertures are located between the anodes and cathodes.

(2) loading a sample that contains a plurality of components, including a biomolecule (such as a protein) of interest, into at least one loading well through at least one loading aperture of the electrophoresis cassette.

(3) applying an electric field between the two electrodes to drive electrophoretic migration of the biomolecule into a collection well, and

(4) removing the liquid containing the biomolecule or band of interest from the collection well through the collection aperture, thereby isolating the biomolecule or band of interest.

In certain illustrative embodiments, such electrophoresis cassettes have at least four walls which are sealed to form a separation chamber surrounded by the walls, and the separation chamber contains an electrophoresis gel matrix which has at least 2 wells therein. In addition, at least one wall of such cassettes has an array of openings (also referred to herein as apertures) for access from outside of the cassette to the wells formed in the electrophoresis gel contained in the separation chamber, or for access from outside of the cassette to the empty separation chamber inside the cassette. Such cassettes also include all the chemical compounds required for driving electrophoresis separations and, in certain embodiments, for enabling visualization of the separated sample bands. In addition, such cassettes can be disposable. In certain embodiments the electrophoresis gel is put into the separation chamber by an outside vendor, while in other embodiment the separation chamber does not contain an electrophoresis gel until an end user puts the electrophoresis gel into the separation chamber before using the electrophoresis cassette.

In certain embodiments, the electrophoresis cassettes can have the electrodes in regions in the cassette which contain liquid buffer. In certain embodiments the regions containing liquid buffers are in the same plane as the electrophoresis gel, while in other embodiments the regions containing liquid buffers are located below or above the plane of the electrophoresis gel. In other embodiments the electrodes are in direct contact with gels, such as electrophoresis gels, which contain ions to facilitate the applied electric field but without the need for liquid buffers. In other embodiments the electrodes are embedded in gels, such as electrophoresis gels, which contain ions to facilitate the applied electric field but without the need for liquid buffers. In other embodiments the electrodes are in indirect contact with gels, such as electrophoresis gels, which contain ions to facilitate the applied electric field but without the need for liquid buffers, such indirect contact can be via another gel material or by simple electrical contact.

Alternative methods for the separation, isolation and collection of a sample component of interest are open systems that use electrophoretic slab gels wherein the slab gel is not immersed in running buffer and is not contained within a cassette. In such a method the slab gel comprises an upper surface and a lower surface wherein the upper surface is not in contact with a liquid, or the slab gel is not immersed in a running buffer used to drive the electrophoretic separation. In such methods, by way of example only, the slab gel is placed in contact with electrodes, either directly or indirectly and an electric field is applied to drive electrophoretic migration of the sample. Indirect contact can be achieved using wicking techniques, wherein the electrodes are placed in buffer tanks which have wicking means between the slab gel and the tank, or indirect contact may be achieved using a gel matrix located between the electrodes and the slab gel. In addition, the slab gels used in such methods contains arrays of loading wells and collection wells as disclosed herein for the electrophoresis cassettes, and although no apertures are used the methods for separation, isolation and collection are the same as those disclosed for the electrophoresis cassettes. There is no fluid communication between the liquid in the collection wells and another liquid, and there is no fluid communication between the liquid in the collection wells and a running buffer used to drive the electrophoretic separation. In addition, the liquid in the collection wells makes no contact with another liquid in the electrophoresis cassette during the loading or the collecting, and there is no liquid above the electrophoresis slab gel in contact with a collection well. Furthermore, the liquid in the collection wells and optionally a liquid in the sample wells are the only liquids within the electrophoresis cassette, and the liquid in the collection wells is isolated from other liquids that optionally are present within the electrophoresis cassette.

Design and Patterns of Wells in Electrophoresis Gels

The non-limiting embodiments disclosed above and illustrated in FIGS. 3-8 utilize a single column with one loading well and at least one collection well. However, other embodiments of the electrophoresis systems, assemblies, cassettes and methods disclosed herein use arrays of wells and apertures wherein multiple loading wells and loading apertures can be used along with multiple collection wells and collection apertures. Such arrays of wells and apertures can be disclosed as being “r×c” arrays, wherein r is the number of rows and c is the number of columns. The number of rows, r, and the number of columns, c, of such arrays are independent of each other and therefore the arrays of wells and apertures used in the methods disclosed herein can be symmetric arrays (where r=c) or asymmetric arrays (where r≠c). Such arrays of wells and apertures can include at least 1 row, at least 2 rows, at least 3 rows, at least 4 rows, at least 5 rows, at least 6 rows, at least 7 rows, at least 8 rows, at least 9 rows, at least 10 rows, at least 11 rows, at least 12 rows, at least 15 rows, at least 20 rows, at least 50 rows, or at least 100 rows each independently in combination with at least 1 column, at least 2 columns, at least 3 columns, at least 4 columns, at least 5 columns, at least 6 columns, at least 7 columns, at least 8 columns, at least 9 columns, at least 10 columns, at least 11 columns, at least 12 columns, at least 15 columns, at least 20 columns, at least 50 columns, or at least 100 columns.

Non-limiting examples of the arrays of wells and apertures used in the methods disclosed herein include, but are not limited to, r×1 arrays, r×2 arrays, r×3 arrays, r×4 arrays, r×5 arrays, r×6 arrays, r×7 arrays, r×8 arrays, r×9 arrays, r×10 arrays, r×11 arrays, r×12, r×13 arrays, r×14 arrays, r×15 arrays, r×16 arrays, r×17 arrays, r×18 arrays, r×19 arrays, r×20 arrays, r×21 arrays, r×22 arrays, r×23 arrays, r×24 arrays, r×25, and r×26 arrays, where r is an integer from 2 to 26.

Other non-limiting examples of the arrays of wells and apertures used in the methods disclosed herein include, but are not limited to, 1×c arrays, 2×c arrays, 3×c arrays, 4×c arrays, 5×c arrays, 6×c arrays, 7×c arrays, 8×c arrays, 9×c arrays, 10×c arrays, 11×c arrays, 12×c, 13×c arrays, 14×c arrays, 15×c arrays, 16×c arrays, 17×c arrays, 18×c arrays, 19×c arrays, 20×c arrays, 21×c arrays, 22×c arrays, 23×c arrays, 24×c arrays, 25×c, and 26×c arrays, where c is an integer from 2 to 26.

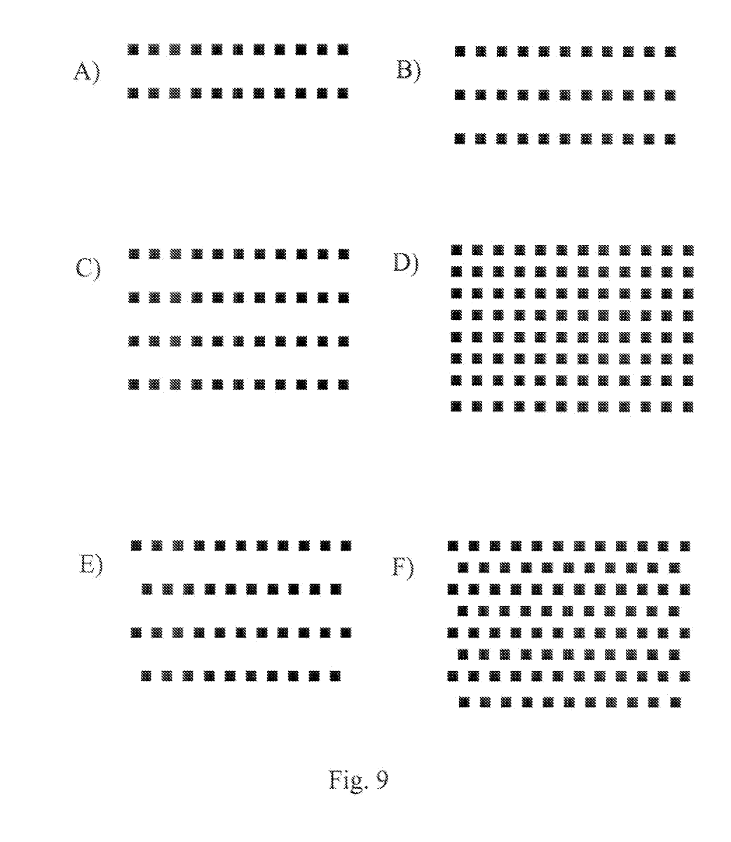

The arrangement of wells and apertures in the arrays of wells used in the methods disclosed herein include, but are not limited to, those illustrated in FIG. 9. Such arrangements can be a checkerboard pattern as shown in FIGS. 9E-9F, wherein the rows are arranged in an alternating staggered format. Alternatively, the arrangement of wells and apertures can be the pattern as shown in FIGS. 9A-9D. The number of wells and apertures in such array patterns can be from 2 to 200, from 2 to 150, from 2 to 96, from 2 to 48, from 2 to 24, or from 2 to 12.

In certain embodiments the spacing between the wells, and the spacing between the apertures, in the rows of the arrays of wells and apertures is equidistant and can range between 5 mm to 10 cm measured from the center of one well to the next well. In certain embodiments the spacing between the wells, and the spacing between the apertures, in the columns of the arrays of wells and apertures is equidistant and can range between 5 mm to 10 cm measured from the center of one well to the next well.

In certain embodiments the spacing between the wells, and the spacing between the apertures, in the rows of the arrays of wells and apertures can increase from left to right in linear increments, with the first spacing in the range between 5 mm to 10 cm measured from the center of the first well to the next well and the increment step in the range between 5 mm to 10 cm. Alternatively, in certain embodiments the spacing between the wells, and the spacing between the apertures, in the rows of the arrays of wells and apertures can decrease from left to right in linear increments, with the first spacing in the range between 5 mm to 10 cm measured from the center of the first well to the next well and the increment in the range between 5 mm to 10 cm.

In certain embodiments the spacing between the wells, and the spacing between the apertures, in the rows of the arrays of wells and apertures can increase from left to right in non-linear increments, with the first spacing in the range between 5 mm to 10 cm measured from the center of the first well to the next well and the increment step in the range between 5 mm to 10 cm. Alternatively, in certain embodiments the spacing between the wells, and the spacing between the apertures, in the rows of the arrays of wells and apertures can decrease from left to right in non-linear increments, with the first spacing in the range between 5 mm to 10 cm measured from the center of the first well to the next well and the increment step in the range between 5 mm to 10 cm.

In certain embodiments the spacing between the wells, and the spacing between the apertures, in the columns of the arrays of wells and apertures can increase from top to bottom in linear increments, with the first spacing in the range between 5 mm to 10 cm measured from the center of the first well to the next well and the increment step in the range between 5 mm to 10 cm. Alternatively, in certain embodiments the spacing between the wells, and the spacing between the apertures, in the columns of the arrays of wells and apertures can decrease from top to bottom in linear increments, with the first spacing in the range between 5 mm to 10 cm measured from the center of the first well to the next well and the increment step in the range between 5 mm to 10 cm.

In certain embodiments the spacing between the wells, and the spacing between the apertures, in the columns of the arrays of wells and apertures can increase from top to bottom in non-linear increments, with the first spacing in the range between 5 mm to 10 cm measured from the center of the first well to the next well and the increment step in the range between 5 mm to 10 cm. Alternatively, in certain embodiments the spacing between the wells and the spacing between the apertures, in the columns of the arrays of wells can decrease from top to bottom in non-linear increments, with the first spacing in the range between 5 mm to 10 cm measured from the center of the first well to the next well and the increment step in the range between 5 mm to 10 cm.

Each well and apertures in an array of wells and apertures can be, independent of the other, circular, semi-circular, square, rectangular, triangular, or oval in shape. The dimensions of different shaped wells can be as follows:
circular wells: diameter between 2 mm to 15 mm; and depth between 1 mm and 6 mm. In certain embodiments, the diameter is between 2 mm to 10 mm; and the depth is between 1 mm and 6 mm. In certain embodiments, the diameter is between 2 mm to 5 mm; and the depth is between 1 mm and 6 mm.
semi-circular wells: radius between 1 mm to 7.5 mm; and depth between 1 mm and 6 mm. In certain embodiments the radius is between 1 mm to 5 mm; and the depth is between 1 mm and 6 mm. In certain embodiments the radius is between 1 mm to 3 mm; and the depth is between 1 mm and 6 mm.
square wells: length and width between 2 mm to 15 mm; and depth between 1 mm and 6 mm. In certain embodiments the length and width are between 2 mm to 10 mm; and the depth is between 1 mm and 6 mm. In certain embodiments the length and width are between 2 mm to 5 mm; and the depth is between 1 mm and 6 mm.
rectangular wells: length between 2 mm to 15 mm; width between 2 mm to 15 mm; and depth between 1 mm and 6 mm. In certain embodiments the length and width are between 2 mm to 10 mm; and the depth is between 1 mm and 6 mm. In certain embodiments the length and width are between 2 mm to 5 mm; and the depth is between 1 mm and 6 mm.
triangular wells: length between 2 mm to 15 mm; height between 2 mm to 15 mm; and depth between 1 mm and 6 mm. In certain embodiments the length is between 2 mm to 10 mm, the height between 2 mm to 10 mm; and the depth between 1 mm and 6 mm. In certain embodiments the length is between 2 mm to 5 mm, the height between 2 mm to 5 mm; and the depth between 1 mm and 6 mm.
oval wells: length between 2 mm to 15 mm; height between 2 mm to 15 mm; and depth between 1 mm and 6 mm. In certain embodiments the length is between 2 mm to 10 mm, the height between 2 mm to 10 mm; and the depth between 1 mm and 6 mm. In certain embodiments the length is between 2 mm to 5 mm, the height between 2 mm to 5 mm; and the depth between 1 mm and 6 mm.

Although the wells can be circular or oval in shape it is preferred that the wells of the invention be square, rectangular, semi-circular or triangular with a substantially flat wall in the direction of electrophoresis, because non-flat walls in the direction of electrophoresis can adversely affect the shape of a sample band during electrophoresis and thereby affect the resolution of separating sample components. In addition, the depth of the wells should be less than the thickness of the electrophoresis gel, wherein the bottom of the wells are formed by the electrophoresis gel and not by the wall of the electrophoresis cassette.

Each well in an array of wells can have, independent of the other wells, a volume ranging from 150 nL to 14 mL. In certain embodiments the volume of each well, independent of other wells can range from 5 μL to 10 mL. In certain embodiments the volume of each well, independent of other wells can range from 5 uL to 1 mL. In certain embodiments the volume of each well, independent of other wells can range from 5 μL to 500 μL. In certain embodiments the volume of each well, independent of other wells can range from 5 μL to 200 μl. In certain embodiments the volume of each well, independent of other wells can range from 5 μL to 100 μL. The larger volume wells, including but not limited to wells having volumes ranging from 500 μL to 14 mL, can be used for the separation, isolation and collection of a component of interest from large sample volumes

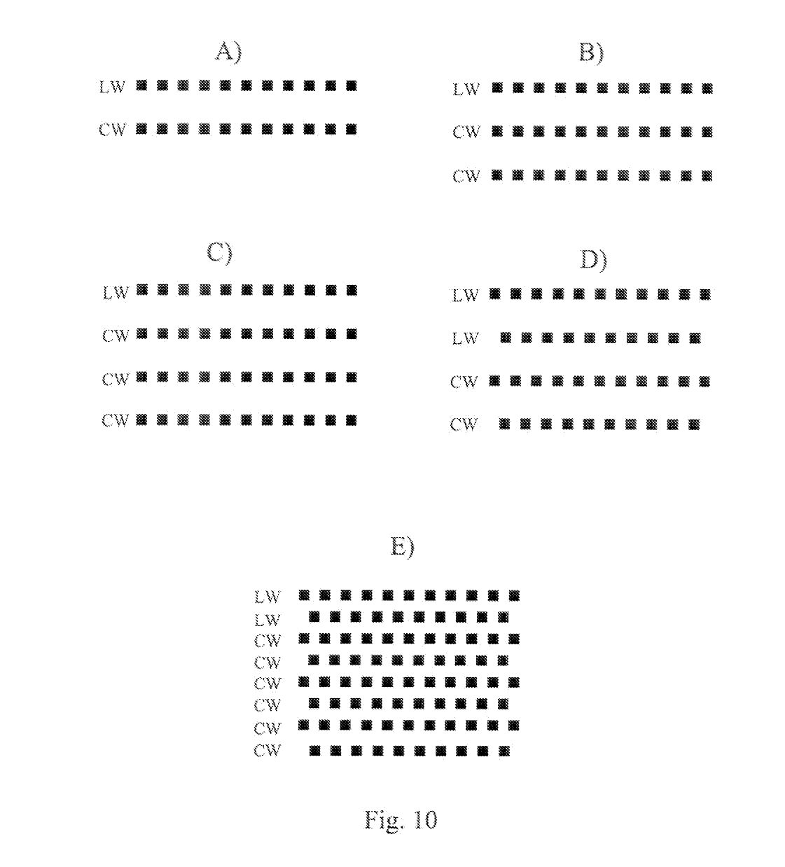

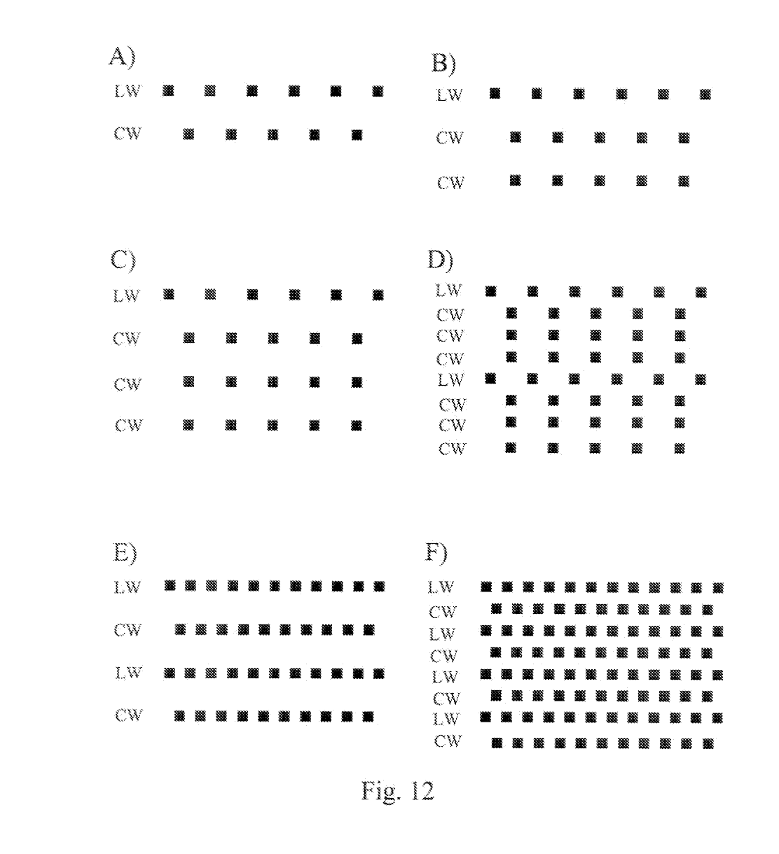

Non-limiting examples of the arrangement of loading wells and apertures and collection wells and apertures of the electrophoresis gel used in the methods disclosed herein are shown in FIG. 10 and FIG. 12.

The polymeric components of the gel cassettes can be made of a polymer which is transparent to visible light, transparent to ultraviolet light, transparent to infra-red light, or transparent to both visible and ultraviolet light. Non-limiting examples of polymers used to make the gel cassettes disclosed herein are styrene acrylonitrile, polycarbonate, polystyrene, acrylic based polymers, polymethyl methacrylate, polyethylene terephthalate, glycol-modified polyethylene terephthalate, polypropylene, Acetel and copolymers thereof. The polymeric components of the gel cassettes may be fabricated using molding techniques, hot embossing methods, casting processes, thermoforming methods, stereolithography processes, machining methods and milling processes. In further or alternative embodiments, such molding techniques include injection molded and compression molding.

As disclosed herein the gel cassettes also includes two electrodes, an anode and a cathode, wherein the array of wells and apertures are located between the electrodes. Such electrodes are used to create an electric field used to drive electrophoretic migration and separation of the components of a sample. The electrodes of the electrophoresis cassette can be electrically conductive metallic material or electrically conductive non-metallic including, but not limited to, platinum, palladium, gold, copper, lead, aluminum, silver, nickel, iron, stainless steel, graphite, or carbon. Alternatively, the electrodes of the electrophoresis cassette can comprise a non-conducting material which is coated with an electrically conductive metal or non-metal including, but not limited to, platinum, palladium, gold, copper, lead, aluminum, silver, nickel, iron, stainless steel, graphite, carbon or combinations thereof.

In addition, the gel cassette can include a second pair of electrodes, which are orthogonal to the first pair of electrodes, with the array of wells located between the first and second pair of electrodes. Such an arrangement of electrodes can allow for 2-dimensional (2D) electrophoretic migration and separation of the components of a sample as disclosed herein.

The array of apertures in the electrophoresis gel cassette has at least one row or column of loading apertures in which a sample can be loaded through an aperture into a corresponding loading well located underneath the loading aperture. In addition, the array of apertures can have at least one row or column of collection apertures in which a separated and isolated sample component, or a purified sample, can be removed from a collection well through a corresponding collection aperture located above the collection well.

In another aspect of the methods described herein, the collection wells are loaded with coated particles prior to, or during, electrophoretic transport of the sample through the electrophoresis gel. Such methods can be used to purify a biomolecule by isolating the biomolecule from a sample. The particles used to isolate a biomolecule using the methods, electrophoresis systems, assemblies, apparatuses and cassettes described herein are coated with a moiety that binds specifically to the biomolecule of interest. Such a moiety can be either covalently or non-covalently attached to the particle surface. In certain embodiments the moiety is adsorbed onto the surface of the particles. The biomolecule specific moiety used in the methods, electrophoresis systems, assemblies, apparatuses and cassettes described herein includes, but is not limited to, oligonucleotides having a specific sequence complementary to the biomolecule of interest, oligonucleotides having a general sequence such as, by way of example only, Poly A, primers, antibodies, specific antibodies, peptides, protein A or G, lectins, receptors, biotin, avidin, streptavidin, gluthatione, His-tag and cellulose binding domains (CBD). In certain embodiments the biolmolecule of interest is isolated by immuno precipitation. In certain embodiments the biolmolecule of interest is isolated from buffers containing salts.

In certain embodiments the particles are removed from the collection wells, while in other embodiments the biomolecule of interest is released from the particle and the solution containing the biomolecule is removed from the collection wells. The isolated biomolecule can then be used in other biological processes, including but not limited to, cloning or ligation, and therefore it is desired that the component of interest is not damaged during the visualization process. In certain embodiments the biochemical process is restriction enzyme cloning, while in other embodiments the biochemical process is high-throughput recombination cloning. In certain embodiments the biochemical process is TOPO® (Invitrogen Corp., Carlsbad) restriction cloning, while in other embodiments the biochemical process is GATEWAY® (Invitrogen Corp., Carlsbad) recombination cloning. In certain embodiments, the efficiencies of such cloning methods are enhanced 10-1000 fold, while in other embodiments the efficiency is enhanced 10-500 fold. In certain embodiments, the efficiencies of such cloning methods are enhanced 10-100 fold. In other embodiments, the isolated biomolecule can be analyzed using sequencing, using mass spectrometrtry, using nucleic acid arrays and/or protein arrays. In other embodiments, the isolated biomolecule can be spotted on an array. In other embodiments, the isolated biomolecule can be used as antigen for immunization.

The particles used to isolate a biomolecule using the methods, electrophoresis systems, assemblies, apparatuses and cassettes described herein can be spherical or toroidal particles or beads. The particles may be glass or polymer beads, and such polymer beads may include, but are not limited to, polystyrene beads, polyacrylamide beads, polymethyl acrylate beads, agarose beads, derivatized cellulose fibers, carboxylated polystyrene beads, polyvinylchloride beads, polymethylacrylate beads, polypropylene beads, latex beads, polytetrafluorethylene beads, or polyacrylonitrile beads. In certain embodiments the size of such particles is between about 10 microns and about 500 microns. In certain embodiments the size of such particles is between about 10 microns and about 250 microns. In certain embodiments the size of such particles is between about 10 microns and about 100 microns. In certain embodiments the size of such particles is between about 10 microns and about 50 microns. In certain embodiments the size of such particles is between about 50 microns and about 500 microns. In certain embodiments the size of such particles is between about 50 microns and about 250 microns. In certain embodiments the size of such particles is between about 50 microns and about 100 microns.

In certain embodiments the biomolecule of interest is released from the biomolecule specific moiety by increasing the pH of the liquid within the collection wells. In other embodiments the biomolecule of interest is released from the biomolecule specific moiety by decreasing the pH of the liquid within the collection wells. In certain embodiments the biomolecule of interest is released from the biomolecule specific moiety by increasing the ionic strength of the liquid within the collection wells. In certain embodiments the biomolecule of interest is released from the biomolecule specific moiety by decreasing the ionic strength of the liquid within the collection wells. In certain embodiments the biomolecule of interest is released from the biomolecule specific moiety by addition of a reducing agent to the liquid within the collection wells. In certain embodiments the biomolecule of interest is released from the biomolecule specific moiety by addition a competition reagent that competes with the biomolecule of interest for binding to the biomolecule specific moiety. Such competition reagents include, but are not limited to, imidazole for His tag immobilized beads, reduced gluthatione for glutathione tagged beads or protease such as factor Xa to cleave away a GST tag.

In certain aspects, no collection well is present in the electrophoresis gel, however, one or more collection apertures are present. In these aspects, a separated sample component can be isolated away from the electrophoresis gel by inserting a collection instrument that is capable of removing a section of a gel accessible through the collection aperture, such as an instrument that is capable of boring holes in the gel and collecting the removed gel slice. Typically, the gel slice can be in the size range and shape disclosed herein for collection wells.

Each loading well of the electrophoresis gel is located at the beginning of an electrophoresis lane, and each loading well is in alignment with at least one collection well located along the electrophoresis lane of the corresponding loading well.

The electrophoresis gel cassettes used in the methods disclosed herein can optionally have a cation ion exchange matrices located between each anode and the electrophoresis gel, and can have an anion ion exchange matrices located between each cathode and the electrophoresis gel. A non-limiting example of a cation exchange material incorporated into the gel cassette is CM-25-120 Sephadex and a non-limiting example an anion exchange material incorporated into the gel cassette is WA-30, both of which are commercially available from Sigma Inc. of St. Louis, U.S.A.

The electrophoresis gel cassettes used in the methods disclosed herein can have the electrophoresis gel already cast in the separation chamber with the wells being optionally occupied by at least one gel comb. The comb or combs are removed to give wells available for use as loading wells and collection wells. Alternatively, the electrophoresis gel cassettes used in the methods disclosed herein can be empty and the electrophoresis gel is cast, using appropriate combs, by the user to create an array of wells available for use as loading wells and collection wells.

In certain embodiments, the electrophoresis gel cassettes used in the methods disclosed herein has at least one marking located on at least one wall of the electrophoresis cassette. Such markings can be located beside the electrophoresis lane in which the bands migrate or the markers are located within the electrophoresis lane in which the bands migrate. Such markings can be used to aid in determining when the sample band of interest will be located in a collection well as disclosed herein.

The electrophoresis gel used in the methods disclosed herein can comprise any material which forms a gel including, but not limited to, synthetic polymers, natural polymers and combinations thereof. Examples of such synthetic polymers includes, but is not limited to, linear polyacrylamide, crosslinked polyacrylamide and polyvinylpyrrolidone. Examples of such natural polymers includes, but is not limited to, polysaccharides such as agarose, carrageenan, and chitosan.

In certain embodiments, the electrophoresis gel used in the methods disclosed herein comprise acrylamide, including by way for example only, acrylamide at a concentration from about 2.5% to about 30%, or from about 5% to about 20%. In certain embodiments, such polyacrylamide electrophoresis gel comprise 1% to 10% crosslinker, including but not limited to, bisacrylamide. In certain embodiments, the electrophoresis gel used in the methods disclosed herein comprises agarose, including by way for example only, agarose at concentration from about 0.1% to about 5%, or from about 0.5% to about 4%, or from about 1% to about 3%. In certain embodiments, the electrophoresis gel used in the methods disclosed herein comprises 2% agarose, while in other embodiments, the electrophoresis gel used in the methods disclosed herein comprises 0.8% agarose. In certain embodiments, the electrophoresis gel used in the methods disclosed herein comprises 1% agarose, while in other embodiments, the electrophoresis gel used in the methods disclosed herein comprises 0.5% agarose. In certain embodiments, the electrophoresis gel used in the methods disclosed herein comprises acrylamide and agarose, including by way for example only, electrophoresis gels comprising from about 2.5% to about 30% acrylamide and from about 0.1% to about 5% agarose, or from about 5% to about 20% acrylamide and from about 0.2% to about 2.5% agarose. In certain embodiments, such polyacrylamide/agarose electrophoresis gel comprise 1% to 10% crosslinker, including but not limited to, bisacrylamide.

In certain embodiments of the methods disclosed herein the electrophoresis gel in the electrophoresis cassette is a gradient gel. In other embodiments of the methods disclosed herein the electrophoresis gel in the electrophoresis cassette is a highly crosslinked gel.

In certain embodiments of the methods disclosed herein the electrophoresis gel in the electrophoresis cassette is a denaturing gels, wherein the gel, sample or gel and sample include a detergent(s), chaotropic agent(s) or combinations thereof. Chaotropic agents include, but are not limited to, sodium trifluoroacetate, sodium perchlorate, sodium iodide, urea, guanidinium chloride and guanidine isothiocyanate. Denaturing detergents include, but are not limited to, sodium dodecyl sulfate (SDS).

In certain embodiments, the electrophoresis cassette and electrophoresis gel used in the methods disclosed herein are the E-PAGE™ cassettes/gels (Invitrogen, Carlsbad) and the E-GEL® cassettes/gels (Invitrogen Carlsbad). In certain embodiments, the E-PAGE™ (Invitrogen, Carlsbad) cassettes/gels have two rows of wells, one row of loading wells and one row of collection wells. In other embodiments, the E-GEL® cassettes/gels (Invitrogen Carlsbad) have two rows of wells, one row of loading wells and one row of collection wells. In certain embodiments, a 0.8% E-GEL® (Invitrogen Carlsbad) cassettes/gels with two rows of wells, one row of loading wells and one row of collection wells, can be used. In other embodiments, a 2% E-GEL® cassettes/gels (Invitrogen Carlsbad) with two rows of wells, one row of loading wells and one row of collection wells, can be used.

The electrophoresis gel buffer used in the method disclosed herein may be any electrophoresis buffer, including but not limited zwitterionic buffers. In certain embodiments the gel buffer has a pH between 5 and 9 at ambient temperature. In certain embodiments the gel buffer has a pH between 6 and 8.5 at ambient temperature. In certain embodiments the gel buffer has a pH between 6 and 8 at ambient temperature. In certain embodiments the gel buffer has a pH between 6 and 7 at ambient temperature. In certain embodiments the gel buffer has a pH between 5 and 9 at 25° C. In certain embodiments the gel buffer has a pH between 6 and 8.5 at 25° C. In certain embodiments the gel buffer has a pH between 6 and 8 at 25° C. In certain embodiments the gel buffer has a pH between 6 and 7 at 25° C.

In certain embodiments the gel buffer comprises a buffer having a pKa between about 5 and about 8.5 at ambient temperature. In certain embodiments the gel buffer comprises a buffer having a pKa between about 6 and about 8.5 at ambient temperature. In certain embodiments the gel buffer comprises a buffer having a pKa between about 6 and about 8 at ambient temperature. In certain embodiments the gel buffer comprises a buffer having a pKa between about 6 and about 7 at ambient temperature. In certain embodiments the gel buffer comprises a buffer having a pKa between about 5 and about 8.5 at 25° C. In certain embodiments the gel buffer comprises a buffer having a pKa between about 6 and about 8.5 at 25° C. In certain embodiments the gel buffer comprises a buffer having a pKa between about 6 and about 8 at 25° C. In certain embodiments the gel buffer comprises a buffer having a pKa between about 6 and about 7 at 25° C.

The electrophoresis gel buffers used in the methods disclosed herein include, but are not limited to, succinate, citrate, borate, maleate, cacodylate, N-(2-Acetamido)iminodiacetic acid (ADA), 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxypropanesulfonic acid (MOPSO), N,N-bis-(hydroxyethyl)-2-aminoethanesulfonic acid (BES), 3-(N-morpholino)-propanesulfonic acid (MOPS), N-tris-(hydroxymethyl)-2-ethanesulfonic acid (TES), N-2-hydroxyethyl-piperazine-N-2-ethanesulfonic acid (HEPES), 3-(N-tris-(hydroxymethyl)methylamino)-2-hydroxypropanesulfonic acid (TAPSO), 3-(N,N-Bis[2-hydroxyethyl]amino)-2-hydroxypropanesulfonic acid (DIPSO), N-(2-Hydroxyethyl)piperazine-N′-(2-hydroxypropanesulfonic acid) (HEPPSO), 4-(2-Hydroxyethyl)-1-piperazinepropanesulfonic acid (EPPS), N-[Tris(hydroxymethyl)methyl]glycine (Tricine), N,N-Bis(2-hydroxyethyl)glycine (Bicine), (2-Hydroxy-1,1-bis(hydroxymethyl)ethyl)amino]-1-propanesulfonic acid (TAPS), N-(1,1-Dimethyl-2-hydroxyethyl)-3-amino-2-hydroxypropanesulfonic acid (AMPSO), tris(hydroxy methyl)amino-methane(Tris), TRIS-Acetate-EDTA (TAE), glycine, bis[2-hydroxyethyl]iminotris[hydroxymethyl]methane(BisTris), or combinations thereof In addition, such gel buffers can include ethylene diamine tetraacetic acid (EDTA).

The concentration of the electrophoresis gel buffers used in the methods disclosed herein is from about 10 mM to about 1 M. In certain embodiments the concentration is between about 20 mM and about 500 mM, and in other embodiments the concentration is between about 50 mM and about 300 mM.

The electrophoretic migration of the sample components can be achieved using constant voltage, pulsed voltage, constant current, pulsed current, constant power or pulsed power. Subsequently, the electric field (V/cm) applied to the electrodes of the electrophoresis cassettes used in the methods disclosed herein can be constant or pulsed. It is understood that the magnitude of the applied voltage, applied current or applied power to achieve the electric fields ranges provided below will vary depending on the dimensions of the electrophoresis cassette and buffer conductivity. By way of example only, the applied voltage can range from 5V to 2000V, and in certain embodiments the applied voltage can range from 5V to 1000V, 5V to 500V, 5V to 250V, or 5V to 100V. By way of example only, the applied current can range from 5 mA to 400 mA, and in certain embodiments the applied current can range from 5 mA to 200 mA, 5 mA to 100 mA, 5 mA to 50 mA, or 5 mA to 25 mA. In one embodiment the applied current can be 60 mA. By way of example only, the applied power can range from 5 mA to 400 mA, and in certain embodiments the applied current can range from 25 mW to 400 W, 25 mW to 100 W, 25 mW to 50 W, or 25 mW to 25 W. In one embodiment the applied current can be 4.5 W. In addition the polarity of the applied voltage (constant or pulsed) can be positive or negative, and the polarity of the applied current (constant or pulsed) can be positive or negative.

In certain embodiments the magnitude of the constant electric field applied is between 1 V/cm and 100 V/cm. In certain embodiments the magnitude of the constant electric field applied is between 1 V/cm and 50 V/cm. In certain embodiments the magnitude of the constant electric field applied is between 1 V/cm and 25 V/cm. In certain embodiments the magnitude of the constant electric field applied is between 1 V/cm and 15 V/cm. In certain embodiments the magnitude of the constant electric field applied is between 1 V/cm and 10 V/cm.

The profile of the pulsed electric field can be a square wave, triangular wave or sine wave, and such profiles can be symmetric or asymmetric. The pulsed electric field is applied to a constant baseline electric field and the magnitude of this baseline electric field is from 0 V/cm to 100 V/cm. In certain embodiments the magnitude of this baseline electric field is from 0 V/cm to 50 V/cm. the magnitude of this baseline electric field is from 0 V/cm to 25 V/cm. the magnitude of this baseline electric field is from 0 V/cm to 10 V/cm. In certain embodiments the magnitude of the pulsed electric field applied in addition to the baseline electric field is between 1 V/cm and 100 V/cm. In certain embodiments the magnitude of the pulsed electric field applied in addition to the baseline electric field is between 1 V/cm and 50 V/cm. In certain embodiments the magnitude of the pulsed electric field applied in addition to the baseline electric field is between 1 V/cm and 25 V/cm. In certain embodiments the magnitude of the pulsed electric field applied in addition to the baseline electric field is between 1 V/cm and 10 V/cm.

For pulsed electric fields which are symmetric square waves the time the pulsed electric field is applied in addition to the baseline electric field (ON) is the same as the time that the pulsed electric field is not applied (OFF). In certain embodiments the ON and OFF times are between 1 ms and 60 seconds. In certain the ON and OFF times are between 1 ms and 40 seconds. In certain the ON and OFF times are between 1 ms and 30 seconds. In certain the ON and OFF times are between 1 ms and 20 seconds. In certain the ON and OFF times are between 1 ms and 10 seconds. In certain the ON and OFF times are between 1 ms and 5 seconds. In certain the ON and OFF times are between 1 ms and 1 second.

For pulsed electric fields which are asymmetric square wave pulsed electric fields, the time the pulsed electric field is applied in addition to the baseline electric field (ON) is not the same as the time that the pulsed electric field is not applied (OFF). In certain embodiments the ON time is independently between 1 ms and 60 seconds, and the OFF time is independently between 1 ms and 60 seconds. In certain embodiments the ON time is independently between 1 ms and 40 seconds, and the OFF time is independently between 1 ms and 40 seconds. In certain embodiments the ON time is independently between 1 ms and 20 seconds, and the OFF time is independently between 1 ms and 20 seconds. In certain embodiments the ON time is independently between 1 ms and 10 seconds, and the OFF time is independently between 1 ms and 10 seconds. In certain embodiments the ON time is independently between 1 ms and 5 seconds, and the OFF time is independently between 1 ms and 5 seconds. In certain embodiments the ON time is independently between 1 ms and 1 second, and the OFF time is independently between 1 ms and 1 second.

For pulsed electric fields which are symmetric triangular waves the voltage ramp rate (V/s) up to the maximum electric field applied is the same as the time that the voltage ramp rate (V/s) down to the baseline electric field applied. In certain embodiments the voltage ramp up and the voltage ramp down are between 10 mV/s and 100 V/s. For pulsed electric fields which are asymmetric triangular waves the voltage ramp rate (V/s) up to the maximum electric field applied is not the same as the time that the voltage ramp rate (V/s) down to the baseline electric field applied. In certain embodiments the voltage ramp up is independently between 10 mV/s and 100 V/s and the voltage ramp down is independently between 10 mV/s and 100 V/s.

For pulsed electric fields which are symmetric sine waves the period and frequency are constant, and the minimum electric field of the sine wave is the same as the baseline electric field applied. For pulsed electric fields which are asymmetric sine waves the period and frequency are modulated, and the minimum electric field of the sine wave is the same as the baseline electric field applied.

In certain embodiments an alternating electric field can be applied once the biomolecule or band of interest is located in the collection well, thereby maintaining the biomolecule or band within the well. In certain embodiments the frequency of the alternating electric field can range from 0.1 Hz to 1 kHz, while in other embodiments the frequency is in the range of 1 Hz to 1 kHz. In certain embodiments the frequency of the alternating electric field can range from 0.5 Hz to 1 kHz, while in other embodiments the frequency is in the range of 0.5 Hz to 0.5 kHz.

In certain embodiments, the electric field is applied using an E-GEL® POWERBASE™ (Invitrogen Carlsbad) power supply, E-GEL® I-BASE™ (Invitrogen Carlsbad) power supply and an E-BASE® (Invitrogen Carlsbad) power supply.

Visualization & Recovery

The electrophoresis gel in the electrophoresis cassette is monitored while performing the methods disclosed herein. This monitoring, also referred to herein as visualization, is used in order to determine the time in which a sample band of interest has migrated into a collection well, plus, in the case of the 2D methods disclosed herein, when to switch from the first electric field to the second electric field. This visualization can be continuous or it can be intermittent.

Visualization of the sample bands in the electrophoresis gel is achieved by illuminating the electrophoresis gel (within the electrophoresis cassette) with light of appropriate wavelength(s) to allow observation of dyes, stains or other indicators associated with the sample bands. In certain embodiments of the visualization methods, used in the separation, isolation, and collection methods disclosed herein, the dyes, stains or other indicators are added to the sample prior to loading into the loading well or loading wells. In other embodiments, the dyes, stains or other indicators are added to the loading well or loading wells prior to addition of the sample to the loading well or loading wells, while in other embodiments the dyes, stains or other indicators are added to the loading well or loading wells after to addition of the sample to the loading well or loading wells. Alternatively, in certain embodiments of the visualization methods, used in the separation, isolation, and collection methods disclosed herein, the dyes, stains or other indicators are added to the electrophoresis gel whereby they become associated with the sample components during electrophoretic migration. In still other embodiments of the visualization methods, used in the separation, isolation, and collection methods disclosed herein, the dyes, stains or other indicators are covalently attached to the sample components.

The systems, dyes and stains used for visualization can be fluorescent or non-fluorescent. Non-limiting examples of the systems, dyes and stains used in the methods disclosed herein are SYBR SAFE™ stains (Invitrogen, Carlsbad), ethidium bromide, methylene blue, crystal violet, SYBR® stains (Invitrogen, Carlsbad), SYBR® Green (Invitrogen, Carlsbad), SYBR® Green I (Invitrogen, Carlsbad), SYBR® Green II (Invitrogen, Carlsbad), SYBR® Gold (Invitrogen, Carlsbad), SYPRO® Ruby (Invitrogen, Carlsbad), SYPRO® Orange (Invitrogen, Carlsbad), SYPRO® Tangerine (Invitrogen, Carlsbad), GELGREEN™ (Biotium, Hayward), GELRED™ (Biotium, Hayward), SEEBLUE® stains (Invitrogen, Carlsbad), LUMIO™ detection systems (Invitrogen, Carlsbad), LUMIO™ Green (Invitrogen, Carlsbad), and LUMIO™ Red (Invitrogen, Carlsbad).

In other embodiments visualization is achieved by silver staining the sample components. In other embodiments visualization is enhanced by adding contrast agents. In other embodiments visualization is enhanced by adding fluorescence enhancing agents. In other embodiments visualization is enhanced by making the cassette or the collection well area a different color or texture in order to get more contrast such that the band in the well is easily viewed. In other embodiments visualization is enhanced by using a sticker on the bottom of the cartridge in order to get more contrast such that the band in the well is easily viewed.

The light used for visualization can be monochromatic or polychromatic. By way of example only, polychromatic light can be white light, UV light or infra-red light, while monochromatic light can be achieved using lasers or Light Emitting Diodes (LED's), or by specific spectral filtering of sources such as white light, UV light or infra-red light. It would be understood that the desired wavelength of such monochromatic light depends on the specific spectral characteristics of the dye or stain used, and the skilled artisan will know the methods to obtain such monochromatic light.

In certain embodiment visualization is performed in a stand alone “light box” in which the electrophoresis cassette is placed during electrophoretic separation of the sample. In such light boxes the electrophoresis cassette can be illuminated from above or below. Monitoring can be achieved using a CCD camera or a video camera, or by direct observation of the user performing the separation, isolation, and collection. In other embodiments of such visualization methods an electrophoresis/monitoring apparatus is used in which the monitoring means (CCD camera or a video camera, or by direct observation) and the means for application of the electric field or fields are combined into one apparatus. In addition, in other embodiments a means for cooling the electrophoresis cassette during the electrophoresis is incorporate. Such cooling can be achieved by a flow of cooled gas, (by way of example, liquid nitrogen), a fan or a Peltier cooler.

In certain embodiments visualization is achieved using a DARK READER® (Clare Chemicals, Dolores) transilluminator or a SAFE IMAGER™ (Invitrogen, Carlsbad) transilluminator. In certain embodiments, visualization is achieved using an E-GEL® POWERBASE™ (Invitrogen Carlsbad) power supply, in which the electrophoresis cassette containing the electrophoresis gel is connected to, placed over a DARK READER® (Clare Chemicals, Dolores) transilluminator. In certain embodiments, visualization is achieved using an E-GEL® POWERBASE™ (Invitrogen Carlsbad) power supply, in which an E-PAGE™ cassette or an E-GEL® cassette (Invitrogen Carlsbad) is connected to, placed over a DARK READER® (Clare Chemicals, Dolores) transilluminator. In certain embodiments, visualization is achieved using an E-GEL® POWERBASE™ (Invitrogen Carlsbad) power supply, in which the electrophoresis cassette containing the electrophoresis gel is connected to, placed over a SAFE IMAGER™ (Invitrogen, Carlsbad) transilluminator. In certain embodiments, visualization is achieved using an E-GEL® POWERBASE™ (Invitrogen Carlsbad) power supply, in which an E-PAGE™ cassette or an E-GEL® cassette (Invitrogen Carlsbad) is connected to, placed over a SAFE IMAGER™ (Invitrogen, Carlsbad) transilluminator.

In certain embodiments, visualization is achieved using an E-GEL® IBASE™ (Invitrogen Carlsbad) power supply, in which the electrophoresis cassette containing the electrophoresis gel is connected to, placed over a DARK READER® (Clare Chemicals, Dolores) transilluminator. In certain embodiments, visualization is achieved using an E-GEL® IBASE™ (Invitrogen Carlsbad) power supply, in which an E-PAGE™ cassette or an E-GEL® cassette (Invitrogen Carlsbad) is connected to, placed over a DARK READER® (Clare Chemicals, Dolores) transilluminator. In certain embodiments, visualization is achieved using an E-GEL® IBASE™ (Invitrogen Carlsbad) power supply, in which the electrophoresis cassette containing the electrophoresis gel is connected to, placed over a SAFE IMAGER™ (Invitrogen, Carlsbad) transilluminator. In certain embodiments, visualization is achieved using an E-GEL® IBASE™ (Invitrogen Carlsbad) power supply, in which an E-PAGE™ cassette or an E-GEL® cassette (Invitrogen Carlsbad) is connected to, placed over a SAFE IMAGER™ (Invitrogen, Carlsbad) transilluminator.

In certain embodiments, visualization is achieved using an E-GEL® IBASE™ (Invitrogen Carlsbad) power supply and an E-GEL® SAFE IMAGER™ (Invitrogen, Carlsbad) real-time transilluminator, in which the electrophoresis cassette containing the electrophoresis gel is connected to the E-GEL® IBASE™ (Invitrogen Carlsbad) power supply and the separation can be monitored in real time or after the separation is complete. In certain embodiments, visualization is achieved using an E-GEL® IBASE™ (Invitrogen Carlsbad) power supply and an E-GEL® SAFE IMAGER™ (Invitrogen, Carlsbad) real-time transilluminator, in which an E-PAGE™ cassette or an E-GEL® cassette (Invitrogen Carlsbad) is connected to the E-GEL® IBASE™ (Invitrogen Carlsbad) power supply.

In certain embodiments, visualization is achieved using an E-BASE® (Invitrogen Carlsbad) power supply, in which the electrophoresis cassette containing the electrophoresis gel is connected to, and epi-illumination to monitor fluorescent dyes or stains or visible dyes or stains. In certain embodiments, visualization is achieved using an E-BASE® (Invitrogen Carlsbad) power supply, in which an E-PAGE™ cassette or an E-GEL® cassette (Invitrogen Carlsbad) is connected to, and epi-illumination to monitor fluorescent dyes or stains or visible dyes or stains. In such embodiments provided above the epi-illumination can be achieved using the light sources provided herein, including but not limited to white light, blue light, lasers, and Light-Emitting Diodes (LED's).

The electrophoresis gel cassette can be electrophoreses and viewed on a cassette electrophoresis base configured for holding a cassette during electrophoresis, in which the electrophoresis base provides electrical connections for supplying power for electrophoretic separation and also includes a power supply. In these aspects of the invention, a cassette electrophoresis base is configured such that when a cassette is positioned in the base, the base is open below the bottom surface of the cassette, such that light can be directed upward from a light source into the cassette. The entire cassette electrophoresis base can be positioned over a light source during or following electrophoresis for viewing separating or separated molecules within the gel that is within the cassette without removing the cassette from the base. Preferably, when a cassette is positioned in the cassette electrophoresis base, the height of the space beneath the cassette, from the bottom-most surface of the base (in the region of the base that supports the cassette at one or more edges of the cassette) to the bottom surface of the cassette, is less than about 10 cm, less than about 5 cm, less than about 3 cm, or less than about 2 cm, or less than 1 cm. In some embodiments, when a cassette electrophoresis base is placed on top of a light source with a flat upper surface, the distance from the upper surface of the light source to the lower wall of the cassette is from 0 to 2 mm, from 2 to 4 mm, from 4 to 6 mm, from 6 to 8 mm, or from 8 to 10 mm.

The power supply base in preferred embodiments has programmable settings, such as for electrophoresis time, current, and/or voltage, and in preferred embodiments the polarity of the electrical current can be reversed by means of a switch or button.

The electrophoresis cassette base preferably incorporates an AC/DC adapter, such that in can be plugged into a standard electrical outlet and the base includes, or can be connected to, a connector, or power cord, that can be plugged into a standard electrical outlet (output from 100-240 VAC, 50/60 Hz). The power output of the power supply base can be in the range of about 5 to about 240 VDC, for example, from 10-240 VDC, or from 20-100 VDC, or about 48 VDC, and in exemplary embodiments has a minimum current output of about 0.4 A, 0.5 A, 0.6 A, 0.7 A, 0.8 A, 0.9 A, or 1 A. The power supply in some exemplary embodiments can change the anode and cathode polarity. In these embodiments, a switch in anode and cathode polarity can be controlled by the user by means of a switch, dial, or button.

The power supply base can be programmed with one or, preferably, more than one, electrophoresis programs. The program(s) can determine the voltage or current supplied during electrophoresis and/or the duration of electrophoresis. In some exemplary embodiments, at least one program is a “reverse” program that allows the user to switch the anode and cathode polarity. The programs in certain embodiments are modifiable by the user, such as by use of buttons provided on a panel of the power supply electrophoresis cassette base. The power supply base preferably also an on/off switch or button, and an LCD display that displays at least one of: the program being run, the time remaining in the electrophoresis run, the voltage, or the current. The power supply electrophoresis cassette base can further include an indicator light, that can, for example, be an LED light, to indicate when the power supply is on, and an alarm that emits a sound to indicate that the electrophoresis run has been completed.

The power supply electrophoresis cassette base preferably includes a program or control switch or button that allow the user to switch the polarity of electrophoresis. In some preferred embodiments, a “reverse” program is included that the user is able to select using control buttons. The reverse program can reverse the polarity for a given period of time, for example, from 15 seconds to 15 minutes, or from 30 seconds to 10 minutes, or from one minute to five minutes. The voltage output during the reverse program can be the same or different from the voltage output used during a standard electrophoretic separation program.

In some exemplary embodiments, the cassette is used for protein, peptide, or nucleic acid molecule or nucleic acid fragment isolation, for example using a cloning cassette that comprises a gel having two or more wells, in which at least at least a first well and a second well of the two or more wells are aligned in a single electrophoresis lane, and the cassette has apertures over the wells for loading a sample in a first well, and extracting a separated fragment from a second well. Such a cloning cassette is described in U.S. Provisional Patent Application 60/824,210, filed Aug. 31, 2006, and incorporated herein by reference in its entirety.

In some exemplary embodiments, when a cassette is positioned on the base, a light source can be inserted into the space in the base that is below the cassette, or the cassette electrophoresis base can be positioned on a light source base that includes a light source, in which the light source is the portion or surface of the light source base from which light is emitted. In these embodiments, the space beneath the cassette (from the bottom-most surface of the cassette electrophoresis base where it contact the surface it rests on, to the bottom wall of the cassette) is at least 2 mm and can be, for example, from 2 to 4 mm, from 4 to 6 mm, from 6 to 8 mm, or from 8 to 10 mm, from 1 cm to 2 cm, from 2 cm to 4 cm, from 4 cm to 6 cm, from 6 cm to 8 cm, from 8 cm to 10 cm, or greater than 10 cm. The light source can be any type of light source that directs light upward (toward a cassette positioned on the base). The light emitted by the light source can be of any wavelength range, for example in the UV, visible, or infrared wavelengths, or a combination thereof. Molecular separation can therefore be viewed as it is occurring during electrophoresis by means of a light source that is part of a light source base positioned underneath the cassette electrophoresis base.

In embodiments in which the cassette electrophoresis base is positioned over a light source base, the light source (light emitting portion) of the light source base occupies at least a portion of the space beneath the cassette in the electrophoresis base, and in some embodiments occupies such as 90% or more, 95% or more, or 97% or more, or essentially all of the open space beneath a cassette positioned in the electrophoresis base. In exemplary embodiments, the cassette electrophoresis base is positioned over a light source base that includes a light source that fits the space in the electrophoresis base that is directly below a cassette positioned in the electrophoresis base.

In certain embodiments of the methods disclosed herein include the electrophoresis system/apparatus includes cassette electrophoresis base that supports a cassette during electrophoresis and comprises a power supply and a light source base that can reversibly engage the cassette electrophoresis base such that light is directed upward into a cassette supported by the cassette electrophoresis base. In preferred embodiments, the light source base is configured such that the size of the light source (the light emitting portion of the light source base) conforms to the size of the opening, or space, in the cassette electrophoresis base to direct light upward into the cassette but does not emit light outside the boundaries of the cassette.

The cassette electrophoresis base can simply be positioned over the light source, or can reversibly engage the light source by any feasible means. For example, in some exemplary embodiments the light source can comprise support or base regions having slots or grooves that can be slidably engaged by the electrophoresis cassette base, or can have one or more guides, tabs, rims, shoulders, pins, bumps, posts, flanges, or snaps, or the base can have one or more guides, tabs, rims, shoulders, slots, grooves, pins, bumps, posts, flanges, or snaps, for guiding the positioning of the base on the light source and/or engaging the light source. In some exemplary embodiments, one or more tabs, rims, shoulders, bumps, posts, pins, or other protrusions on one or more lower surfaces of the cassette electrophoresis base fit into one or more holes, slots, depressions, or guides on one or more upper surfaces of the light source base to position the cassette electrophoresis base on the light source base. In some exemplary embodiments, one or more tabs, rims, shoulders, bumps, posts, pins, or other protrusions on one or more upper surfaces of the light source base fit into one or more holes, slots, depressions, or guides on one or more lower surfaces of the cassette electrophoresis base to position the cassette electrophoresis base on the light source base.

The light source base can include a electrical connector (power cord) separate from that of the cassette electrophoresis base and an on/off switch or button separate from that of the cassette electrophoresis base. In some preferred embodiments, the light source is a visible light source. In some preferred embodiments, the light source is an LED light source.

The light source can be any of the light sources described in detail herein. In preferred embodiments, the light source emits visible light that can transmit through the bottom of the cassette. The light source can optionally include a filter that filters light emitted by the light source as described herein. In some embodiments, the bottom wall of a cassette used in a cassette electrophoresis base can filter light emitted by the light source. Preferably, a gel comprises a dye that binds biomolecules (e.g., proteins, peptides, or nucleic acid molecules), or samples include a dye when they are loaded in the gel. Preferably a dye used to label biomolecules is a fluorescent dye that absorbs light of a wavelength that is transmitted through the bottom wall of the cassette and emits light of a wavelength that can transmit through the upper wall of the cassette. (The upper wall of the cassette can optionally include a filter to filter out light of wavelengths that are not emitted by the excited fluorophore dye. In an alternative, a viewer can use filtered glasses or a camera or imager that includes a filter for viewing or imaging the gel.) Examples of dyes, light sources, and filters that can be used for visual detection of electrophoresing biomolecules such as nucleic acids and proteins are described herein.

In certain embodiments of the methods described herein, the electrophoresis system for viewing and running an electrophoresis gel and for isolating biomolecules such as nucleic acid molecules, nucleic acid fragments, proteins, or peptides, comprises a base for positioning the cassette during electrophoresis (also referred to as an “cassette electrophoresis base”) that comprises at least two electrical contact points for contacting electrodes of a cassette positioned on the base and at least one connector that can connect to a power source, such as an electrical outlet; and further includes a light source base configured to reversibly engage the cassette electrophoresis base. The cassette electrophoresis base is therefore a power supply on which a cassette can be positioned during electrophoresis, in which the power supply can supply a set current through the cassette and/or provide a set voltage across the electrodes of a cassette positioned on the base. The cassette electrophoresis base is configured to engage a cassette along at least one edge of the cassette. The cassette electrophoresis base is configured such that when an electrophoresis cassette is positioned in the base, there is a space underneath the cassette in the region of the cassette in which electrophoretic separation occurs. (That is, underneath the region of the cassette corresponding to the region of the gel in which molecular separation occurs, the base does not have any structures, but rather is open, such that there are no parts of the base that block or obscure transmission of light upward into the cassette from a light source positioned underneath the cassette).

The light source base includes a light source that, when positioned under the electrophoresis base, directs light upward into a cassette positioned in the electrophoresis base. The light source base includes a power cord and preferably also includes an on/off switch.

The electrophoresis cassette base of the electrophoresis running/viewing system also has an on/off switch and preferably one or more additional switches, buttons, or dials that control one or more of the voltage or current output, the programmed duration of voltage or current output, the elapsed time of voltage or current output and/or the polarity of the current. The base/power supply preferably also has a display panel, such as a liquid crystal display (LCD) panel or an LED display panel that communicates at least one of elapsed time of an electrophoresis run and the voltage or current output.

In other embodiments, visualization is achieved using a system which combines both the power supply to drive electrophoresis and a visualization system into a single integrated unit (as described, for example, in U.S. provisional application. By way of example only, in certain embodiments visualization is achieved using an E-GEL® IBASE™ (Invitrogen, Carlsbad) power supply which has been integrated into a visualization system disclosed herein.

The gel cassette in these aspects can be electrophoresed and viewed on a base for viewing and running an electrophoresis gel that comprises a base for positioning the cassette during electrophoresis (also referred to as an “integrated electrophoresis cassette base”) that comprises at least two electrical contact points for contacting electrodes of a cassette positioned on the base and at least one connector that can connect to a power source, and also comprises at least one light source that can emit light into a cassette positioned on the base. The integrated electrophoresis cassette base is therefore a combined power supply/light source on which a cassette can be positioned during electrophoresis, in which the power supply can supply a set current through the cassette and/or provide a set voltage across the electrodes of a cassette positioned on the base. The electrophoresis cassette base has an on/off switch and preferably one or more additional switches, buttons, or dials that control one or more of the voltage or current output, the programmed duration of voltage or current output, and/or the elapsed time of voltage or current output. In some preferred embodiments, the cassette electrophoresis base with integrated light source has one or more controls that reverse the polarity of the anode and cathode, such that electrophoresis can occur in one direction (typically proceeding from the wells toward the anode), and subsequently the direction of electrophoresis can be reversed. The base/power supply preferably also has a display panel, such as a liquid crystal display (LCD) panel or an LED display panel that communicates at least one of elapsed time of an electrophoresis run and the voltage or current output.

In certain embodiments, intermittent visualization is achieved using electrophoresis cassette which includes markings as disclosed herein. Since such markings can be used to determine when a sample band of interest will be located in a collection well, it is not necessary to continuously monitor the gel cassette. In such methods the sample band of interest is observed to pass a marking located before the collection well. The time required for the sample band of interest to migrate into the next collection well can be calculated by dividing the distance the marking is from the collection well by the migration rate of the band. The sample component can then be removed from the collection well.

In certain embodiments the sample component of interest is used in other biological processes, including but not limited to, cloning or ligation, and therefore it is desired that the component of interest is not damaged during the visualization process. In certain embodiments the biochemical process is restriction enzyme cloning, while in other embodiments the biochemical process is high-throughput recombination cloning. In certain embodiments the biochemical process is TOPO® (Invitrogen Corp., Carlsbad) restriction cloning, while in other embodiments the biochemical process is GATEWAY® (Invitrogen Corp., Carlsbad) recombination cloning. In certain embodiments, the efficiencies of such cloning methods are enhanced 10-1000 fold, while in other embodiments the efficiency is enhanced 10-500 fold. In certain embodiments, the efficiencies of such cloning methods are enhanced 10-100 fold. In the case of DNA, such damage can occur from the light used for illumination (by way of example only, UV light) or from the dye used to aid in visualization (by way of example only, ethidium bromide). In certain embodiments visible light, rather than UV light, is used to minimize and/or eliminate damage to the sample component of interest. In other embodiments visible light and SYBR safe dyes are used for visualization and to minimize and/or eliminate damage to the sample component of interest.

Recovery of the sample component from the collection well or wells can be achieved using manual micropipettors, including single-tip or multi-tip pipetors, or the component can be removed from the collection well(s) using robotic pipetting systems.

In another aspect provided herein are kits that can be used with the electrophoresis systems, assemblies, apparatuses and methods described herein. Such kits include a multi-well electrophoresis gel cassette as disclosed herein and a power supply. In certain embodiments, such kits include a electrophoresis gel cassette having two rows of wells as disclosed herein and a power supply. In certain embodiments such electrophoresis gel cassettes contain agarose, while in other embodiments such electrophoresis gel cassettes contain polyacrylamide. In other embodiments such electrophoresis gel cassettes contain agarose and polyacylamide.

It will be appreciated by persons skilled in the art that the present invention is not limited to what has been particularly shown and disclosed hereinabove. The invention will be further clarified by a consideration of the following examples, which are intended to be purely exemplary of the invention and not to in any way limit its scope.

The following examples are intended to illustrate but not limit the invention disclosed herein.

### EXAMPLES

### Example 1

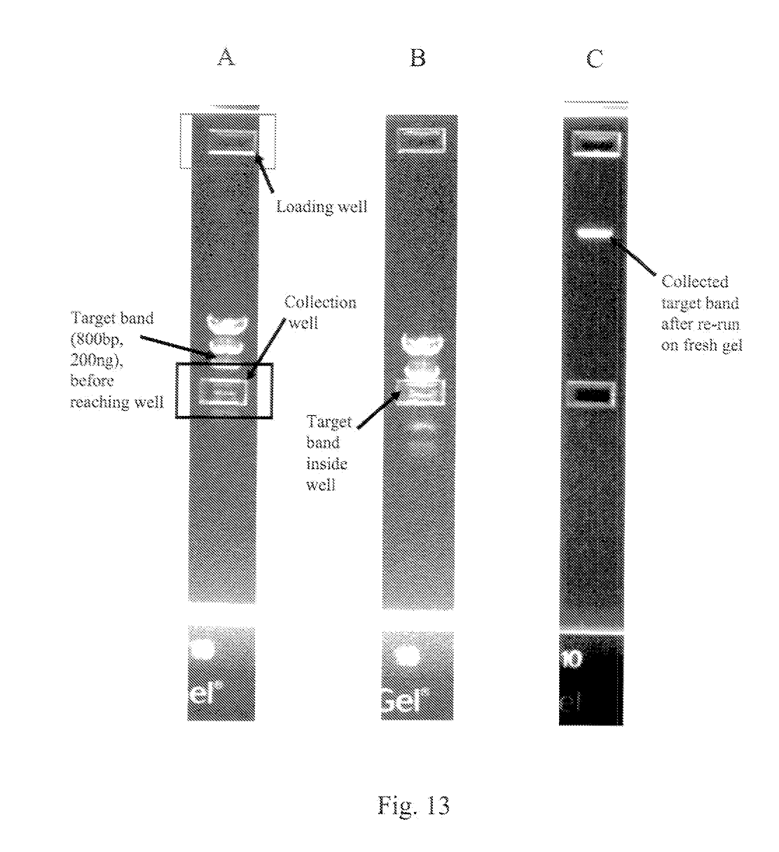

Using an E-GEL® 0.8% double comb cassette with SYBR SAFE™ gel (Invitrogen, Carlsbad), 10 μL of low mass oligonucleotide ladder (Cat. #10068-013 Invitrogen Carlsbad), diluted in water to a final volume of 20 μL, was loaded into each well of the first row of wells and 30 μL of water was added to each well in the second row of wells. The gel was run using an E-GEL® POWERBASE™ (Invitrogen, Carlsbad) power supply placed over a DARK READER® (Clare Chemicals, Dolores) transilluminator. After 15 minutes the DARK READER® (Clare Chemicals, Dolores) transilluminator was turned on in order to monitor the progression of the bands of the separating mass ladder components. FIG. 13 shows the images taken for the isolation and collection of an 800 bp oligonucleotide (fourth band) of interest. In FIG. 13A the first band (100 bp), second band (200 bp) and third band (400 bp) were observed to pass through the collection well of the second row of wells. When the third band was observed to exit the collection well and the fourth band was observed to enter the collection well (FIG. 13B), the electric field was continued for another 2 minutes and then the electric field was turned off. The liquid containing the fourth band (800 bp) was collected from the collection well using a micropipettor. The collected liquid was then run on another E-GEL® cassette (2% double comb containing ethidium bromide) (Invitrogen, Carlsbad) which demonstrated that only one band, with no contaminating bands, was obtained (FIG. 13C). The intensity of the collected band in the E-GEL® cassette (2% double comb containing ethidium bromide) (Invitrogen, Carlsbad) was almost the same intensity as that observed for original mass ladder run on an E-GEL® cassette (2% double comb containing ethidium bromide) (Invitrogen, Carlsbad) which indicated minimal loss of sample.

### Example 2

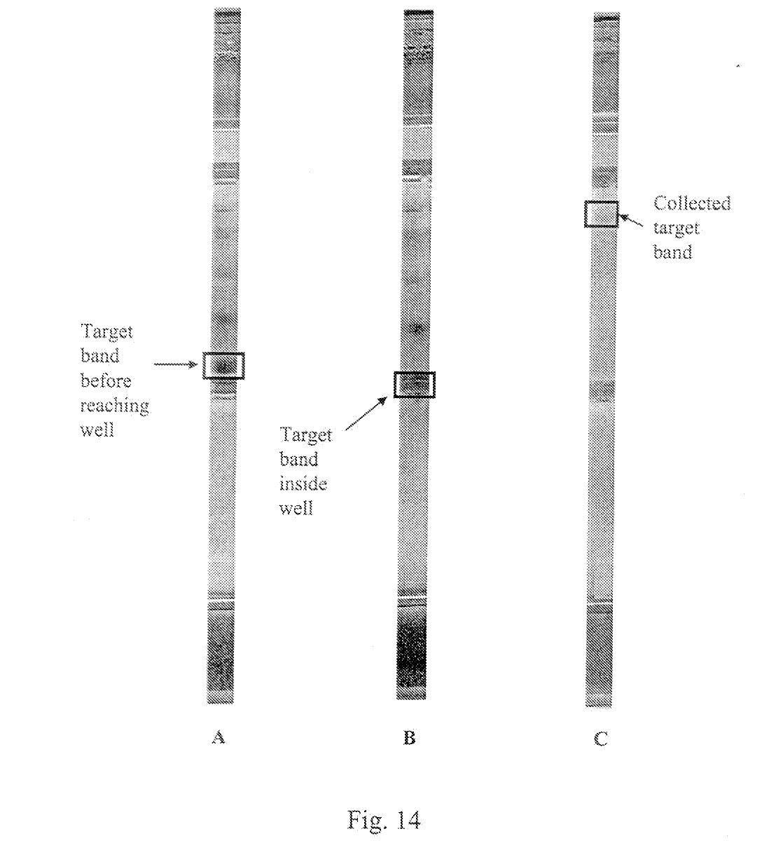

Using an E-PAGE™ 48 8% gel cassette, 15 μl of E-PAGE™ SEEBLUE® (Invitrogen, Carlsbad) pre-stained marker (Cat. #LC5700 Invitrogen, Carlsbad) were loaded into each well of the first row of wells and 20 μl of water were loaded into each well in the second row of wells. The gel was run using an E-BASE® (program EP) (Invitrogen, Carlsbad) power supply. FIG. 14A shows an image taken after 33 minutes of run, when the indicated band (˜21 kD) reached just the edge of the next well, at this stage more water were loaded to the second well (additional ˜10 ul) and the gel was run for 3 more minutes. At this point the indicated band was in the well (FIG. 14B). The run was stopped and the well content was collected using a micropipettor. The collected liquid was run on another E-PAGE™ gel cassette (Invitrogen, Carlsbad) to show one band (FIG. 14C).

### Example 3

Using a 0.8% E-Gel Clonewell cassette (Invitrogen, Carlsbad) cast with no DNA stain inside (a native no size separation gel), a sample containing cells lysate with recombinantly expressed fusion protein containing GST is loaded into the first row of wells and 30 μl of a slurry of agarose beads with immobilized gluthatione is loaded into the second row of wells. The gel is run using an E-GEL® IBASE™ (Invitrogen Carlsbad) power supply for about 30 minutes allowing only the GST tagged protein to bind to the beads, while the other components of the sample migrate through the collection well, or do not migrate in the direction of the GST tagged protein or remain in the loading wells. The power supply is then stopped, and either the pH in the collection wells is lowered or a competitive buffer containing reduced gluthatione is added to the collection wells, or a protease such as factor Xa is added to cleave away the GST tag, thereby releasing the purified protein into the liquid in the well. The liquid containing the purified protein is collected using a micropipettor and the beads are left in the collection well.

### Example 4

Using a 0.8% E-Gel Clonewell cassette, a sample containing a mixture of denatured PCR reaction products is loaded into the first row of wells and 30 μl of a slurry of agarose beads immobilized with a sequence specific oligonucleotide is loaded in the second row of wells. The gel is run for 30 minutes using an E-GEL® IBASE™ (Invitrogen Carlsbad) power supply and the run is monitored over an E-GEL® SAFE IMAGER™ (Invitrogen, Carlsbad) transilluminator to monitor the migration of all DNA bands through the second row of wells. At this point only the band with complementary sequence is bound to the beads and the run is stopped. Salts, chaotropic agents or high pH are added to the well to elute to bound DNA, and the liquid containing the eluted DNA is collected using a micropipettor.

While the invention has been disclosed in connection with illustrative embodiments, it is not intended to limit the scope of the invention to the particular form set forth, but on the contrary, it is intended to cover such alternatives, modifications, and equivalents as may be included within the spirit and scope of the invention as defined by the appended claims.

It should be understood that the foregoing description is only illustrative of the invention. Headings are for convenience only and are not intended to limited disclosure falling under a heading to only that heading. Various alternatives and modifications can be devised by those skilled in the art without departing from the invention. Accordingly, the present invention is intended to embrace all such alternatives, modifications and variances which fall within the scope of the appended claims.

All publications, patents and patent applications mentioned in this specification are herein incorporated by reference in their entirety to the same extent as if each individual publication, patent or patent application was specifically and individually indicated to be incorporated by reference.

What is claimed is:

1. A method for isolating a biomolecule from an electrophoresis gel comprising:
a) obtaining a closed electrophoresis cassette comprising:
i) a separation chamber having walls, wherein at least one of the walls comprises at least one row of loading apertures and at least one row of collection apertures;
ii) an electrophoresis gel contained within the separation chamber;
iii) at least one row of loading wells and at least one row of collection wells within the electrophoresis gel, wherein each loading well is accessible through a loading aperture and each collection well is accessible through a collection aperture, and each loading well is aligned with at least one collection well in an electrophoresis lane, and wherein at least one collection well is filled with a liquid, and
iv) at least two electrodes comprising at least one anode and at least one cathode, wherein the rows of wells and apertures are located between the anodes and cathodes;
b) loading a sample comprising a biomolecule into at least one loading well through at least one loading aperture of the electrophoresis cassette;
c) applying an electric field between the two electrodes to drive electrophoretic migration of the biomolecule into a collection well, and
d) removing the biomolecule from the collection well through the collection aperture, thereby isolating the biomolecule.

2. The method of claim 1, wherein at least one collection well is filled with water or buffer.

3. The method of claim 1, wherein the closed electrophoresis cassette is not immersed in a running buffer used to drive the electrophoretic separation.

4. The method of claim 1, further comprising reloading the collection well with a liquid after the collection step (d).

5. The method of claim 4, further comprising reapplying the electric field between the two electrodes to drive electrophoretic migration of a second biomolecule into the collection well.

6. The method of claim 5, further comprising collecting the second biomolecule from the collection well through the collection aperture.

7. The method of claim 1, further comprising reversing the electric field polarity.

8. The method of claim 1, further comprising monitoring the location of the biomolecule during application of the electric field.

9. The method of claim 9, wherein the monitoring does not damage the biomolecules in the sample.

10. The method of claim 10, wherein the monitoring comprises illuminating with white light, blue light or visible light.

11. The method of claim 9, wherein the monitoring comprises detecting fluorescence.

12. The method of claim 1, further comprising loading a standard comprising a plurality of known molecular markers.

13. The method of claim, 1 further comprising monitoring the biomolecule migrating past a marking located on at least one wall of the electrophoresis cassette.

14. The method of claim 1, wherein the electrophoresis cassette further comprises a dye.

15. The method of claim 1, wherein the electrophoresis gel comprises agarose.

16. The method of claim 1, wherein the electrophoresis gel comprises polyacrylamide, or both agarose and polyacrylamide.

17. The method of claim 1, wherein the biomolecule is DNA or RNA.

18. The method of claim 1, wherein the biomolecule is s a peptide or a protein.

19. The method of claim 1, further comprising loading the electrophoresis cassette into a device comprising a means for applying the electric field and a means for monitoring the plurality of bands during electrophoretic transport of the bands.

20. The method of claim 1, further comprising using the biomolecule in a biochemical process.

21. The method of claim 19, wherein the biochemical process is a cloning reaction.

22. The method of claim 20, wherein the cloning reaction is restriction enzyme cloning.

23. The method of claim 20, wherein the cloning reaction is recombination cloning.

24. An electrophoresis cassette for isolating a biomolecule from an electrophoresis gel comprising:
i) a separation chamber having walls, wherein at least one of the walls comprises at least one row of loading apertures and at least one row of collection apertures;
ii) the electrophoresis gel contained within the separation chamber;
iii) at least one row of loading wells and at least one row of collection wells within the electrophoresis gel, wherein each loading well is located underneath a loading aperture and each collection well is located underneath a collection aperture, and each loading well is aligned with at least one collection well in an electrophoresis lane, and wherein the collection wells are filled with a liquid;
iv) at least two electrodes comprising at least one anode and at least one cathode, wherein the rows of wells and apertures are located between the anode and cathode; and
v) at least one marking on the wall comprising the at least one row of loading apertures and the at least one row of collection apertures, wherein the at least one marking is located between the at least one row of loading apertures and the at least one row of collection apertures.

25. A kit comprising an electrophoresis cassette for isolating a biomolecule from an electrophoresis gel and a power supply, wherein the electrophoresis gel comprises:
i) a separation chamber having walls, wherein at least one of the walls comprises at least one row of loading apertures and at least one row of collection apertures;
ii) the electrophoresis gel contained within the separation chamber;
iii) at least one row of loading wells and at least one row of collection wells within the electrophoresis gel, wherein each loading well is located underneath a loading aperture and each collection well is located underneath a collection aperture, and each loading well is aligned with at least one collection well in an electrophoresis lane, and wherein the collection wells are filled with a liquid;
iv) at least two electrodes comprising at least one anode and at least one cathode, wherein the rows of wells and apertures are located between the anode and cathode; and
v) at least one marking on the wall comprising the at least one row of loading apertures and the at least one row of collection apertures, wherein the at least one marking is located between the at least one row of loading apertures and the at least one row of collection apertures.
